(19) United States
(12) Patent Application Publication
(10) Pub. No.: US 2011/0312927 A1
(43) Pub. Date: Dec. 22, 2011

(54) Progesterone Containing Oral Dosage Forms and Related Methods

(76) Inventors: Satish Kumar Nachaegari, Salt Lake City, UT (US); Chandrashekar Giliyar, Salt Lake City, UT (US); Chidambaram Nachiappan, Sandy, UT (US); Linus Fonkwe, West Valley City, UT (US); Mahesh Patel, Salt Lake City, UT (US); Srinivasan Venkateshwaran, Salt Lake City, UT (US)

(21) Appl. No.: 12/819,125

(22) Filed: Jun. 18, 2010

(51) Int. Cl. A61K 31/57; A61P 15/00; A61P 15/08

(52) U.S. Cl. 514/177

(57) ABSTRACT

The present invention provides for progesterone containing pharmaceutical oral dosage forms and related methods. The oral dosage forms can each include an amount of progesterone as well as a pharmaceutically acceptable carrier. The oral dosage forms can be formulated to have at least one of the following characteristics: the oral dosage form produces an pregnane metabolite mean blood plasma level of less than about 1000 nmol/L; the oral dosage form produces an pregnane metabolites mean blood plasma level, after administration of single dose of progesterone composition, such that the ratio of pregnane metabolite level to parent progesterone level of less than 10:1; has a dissolution rate in vitro, when measure using a USP Type-1 dissolution apparatus in 900 mL of deionized water with 2.0% (w/v) of sodium lauryl sulfate at 100 rpm, such that the oral dosage form releases at least 10 wt % of the progesterone within the first 30 minutes and/or releases less than 45 wt % in the first 4 hours; and the oral dosage form produces a ratio of mean plasma progesterone AUC to the amount of progesterone administered of more than 1.5×10⁻⁶ hr/mL:1

### FIELD OF THE INVENTION

The present invention relates to progesterone compositions, oral dosage forms and associated methods. Accordingly, this invention involves the fields of chemistry, pharmaceutical sciences, medicine and other health sciences.

### BACKGROUND OF THE INVENTION

Progesterone also known as P4 (pregn-4-ene-3,20-dione) is a C-21 steroid hormone involved in the female menstrual cycle, pregnancy (supports gestation) and embryogenesis of humans and other species. Progesterone belongs to a class of hormones called progestogens, and is the major naturally occurring human progestogen. Progesterone has been used in a variety of therapies including the treatment of endometrial hyperplasia in non-hysterectomized postmenopausal women who are receiving conjugated estrogens tablets, secondary amenorrhea, and pregnancy support in Assisted Reproductive Technology (ART) cycles such as In-vitro Fertilization (IVF) and to control anovulatory bleeding.

Orally-administered progesterone undergoes several successive metabolic steps in the gut, intestinal wall, and liver. The first step is the contact with intestine bacteria which has 5β-reductase activity, then with the intestinal wall, predominantly the upper gastro intestinal wall which has 5α-reductase activity and also initiates conjugation of steroids with glucuronic acid. The second step is the contact with liver enzymes after circulation in the portal vascular systems. Liver cells in women express mainly 5β-reductase, 3α and 20α-hydroxylase activities. 3α-OH-5α-pregnan-20-one is known as allopregnanolone and 3α-OH-5,3-pregnan-20-one is known as pregnanolone. Both of the metabolites can be collectively addressed as “pregnane” metabolites. Pregnane metabolites are neurosteroids and are active agonists on the GABA_{A} receptor unlike progesterone. High doses of GABA_{A} receptor agonists such as pregnane metabolites induce dizziness, sedation, hypnosis, and anxiolysis, and are antiepileptic. Therefore, reduced level of pregnane metabolites provides acceptable progesterone therapy without significant adverse events such as sedation, dizziness and hypnosis.

### SUMMARY OF THE INVENTION

The present invention provides for progesterone containing pharmaceutical oral dosage forms and related methods. The oral dosage forms can each include an amount of progesterone as well as a pharmaceutically acceptable carrier. The oral dosage forms can be formulated to have at least one of the following characteristics: the oral dosage form produces a pregnane metabolite mean blood plasma level, after administration of single dose of progesterone composition, of less than about 1000 nmol/L; the oral dosage form produces pregnane metabolite to progesterone mean ratio blood plasma level of less than about 10:1; has a dissolution rate in vitro, when measured using a USP Type-1 dissolution apparatus in 900 mL of deionized water with 2.0% (w/v) of sodium lauryl sulfate at 100 rpm, such that the oral dosage form releases at least 10 wt % of the progesterone in the first 30 minutes or that the oral dosage form releases less than about 45 wt % of the progesterone in the first 4 hours; and the oral dosage form produces a ratio of mean plasma progesterone AUC to the amount of progesterone administered of more than 1.5×10⁻⁶ hr/mL:1. The oral dosage form can be designed, delayed or enteric coated, for targeted delivery to skip the drug release in upper gastrointestinal tract.

The present invention provides specific uses of the compositions and associated methods in pregnancy support that includes progesterone supplementation in the early luteal phase with assisted reproductive technology for embryo impregnation and retention. It can also be given during natural cycles to treat a “luteal phase defect” or to treat a miscarriage (losing fetus at <23 weeks gestation) including early pregnancy loss or clinical spontaneous abortion. The present invention also provides a mechanism for supplementing asymptomatic and symptomatic females requiring pregnancy support with prior obstetrical history of premature birth or with shortened cervix starting mid second trimester (gestational age 16-24) with oral progesterone as a treatment for the prevention of premature (<37 weeks) birth and improving neonatal outcomes including fetal neuro-protection.

In yet a further embodiment, a method of delaying rise of post luteal fetal fibronectin levels in vaginal secretion of at least 50 ng/ml, in a woman that is at least 16 weeks pregnant is encompassed by the present invention. The method includes orally daily administering to the female requiring pregnancy support at least 50 mg/day of progesterone.

The present invention also includes a method of reducing or preventing adverse side effects, including dizziness or sedation associated with the oral administration of progesterone. The method can include administering an oral dosage form as recited herein to a subject. The reduction in dizziness is measured as compared to a dosage form containing micronized progesterone suspended in peanut oil and which provides equivalent progesterone AUC values.

In yet a further embodiment, a method of treating or preventing, or reducing or minimizing the likelihood of, preterm birth, preterm labor, or miscarriage is provided, including incidents characterized fully or in part due to a rise in fetal fibronectin. The method can include administering an oral dosage form as recited herein to a subject in thereof.

### BRIEF DESCRIPTION OF DRAWINGS

FIG. 1 is a plot of the in vitro release profile of progesterone containing oral dosage forms in accordance with certain embodiments of the present invention compared to a dosage form containing 100 mg micronized progesterone suspended in edible oil (Prometrium®).

FIG. 2 is a plot of the in vitro release profile of progesterone containing oral dosage forms in accordance with certain embodiments of the present invention.

FIG. 3 is a plot of the blood plasma concentrations of several subjects receiving oral administration of progesterone oral dosage forms in accordance with certain embodiments of the present invention in both the fed and the fasted state.

FIG. 4 is a plot of the fetal fibronectin levels in asymptomatic women requiring pregnancy support receiving various levels of oral progesterone administration using certain embodiments of the present invention. A control of no progesterone administration is also shown.

FIG. 5 is a plot of the in vitro release profile of progesterone containing oral dosage forms in accordance with certain embodiments of the present invention

### DETAILED DESCRIPTION

Before the present oral dosage forms and methods for the delivery and use of progesterone are disclosed and described, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

### Definitions

As used herein, “drug,” “active agent,” “bioactive agent,” “pharmaceutically active agent,” “therapeutically active agent” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation is intended to include the active agent per se, as well as pharmaceutically acceptable salts, esters or compounds significantly related thereto, including without limitation, prodrugs, active metabolites, isomers, and the like.

As used herein, the term “recurrent” is used to refer repeat or re occurrence of at least one incidence like “miscarriage”, “preterm birth” or any like medical situation in reference with or without same partner, with or without previous live birth.

As used herein, the term “treatment” when used in conjunction with the administration of progesterone, refers to the administration of progesterone to subjects who are either asymptomatic or symptomatic. In other words, “treatment” can both be to reduce or eliminate symptoms associated with a condition or it can be prophylactic treatment, i.e. to prevent the occurrence of the symptoms. Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Furthermore, the term “dosage form” can include one or more formulation(s) or composition(s) provided in a format for administration to a subject. When any of the above terms is modified by the term “oral” such terms refer to compositions, formulations, or dosage forms formulated and intended for oral administration to subjects.

As used herein, “carrier” or “pharmaceutically acceptable carrier” refers to a substance with which a drug may be combined to achieve a specific dosage formulation for delivery to a subject. In the some aspects of the present invention, the carriers used may or may not enhance drug delivery. Further, the carrier, or at least a portion thereof must be suitable for administration into a subject along with the drug.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

In one specific aspect, a subject is a human. In another aspect, the subject is a female. In yet another aspect, the oral dosage form of the current invention is for a female requiring pregnancy support. In another aspect, a female can be 30 years or more in age.

The term “oral administration” represents any method of administration in which an active agent can be administered by swallowing, chewing, or sucking or drinking an oral dosage form. Such solid or liquid oral dosage forms are traditionally intended to substantially release and or deliver the active agent in the gastrointestinal tract beyond the mouth and/or buccal cavity. Examples of solid dosage forms include conventional tablets, multi-layer tablets capsules, caplets, etc., which do not substantially release the drug in the mouth or in the oral cavity.

As used herein, the terms “release”, “release rate” ‘”, are used interchangeably to refer to the discharge or liberation of a substance, including without limitation a drug, from the dosage form into a surrounding environment such as an aqueous medium either in vitro or in vivo.

The term “controlled release,” “sustained release,” “customized release,” “pulsatile release,” “targeted release,” “modified release,” “delayed release” and “extended release” are used interchangeably and refer to release of active agent or agents from a dosage form or at least one of its components (formulations and/or compositions) into the target environment or medium over a period of time that is at least 10% slower with respect to the first 25% of the released active agent than the first 25% of the released active agent from an equivalent dose immediate release (IR) dosage form that release at least 95% drug in the first 30 minutes. It is noteworthy that delayed release can be delayed immediate release or delayed sustained release. In one embodiment, the “controlled release,” “sustained release,” “customized release,” “pulsatile release,” “targeted release,” “modified release,” delayed release,” “extended release,” systems or compositions can provide for a release of the active agent or agents from the dosage form into the target environment or medium over a period of time that is at least 20% slower with respect to the first 25% of the released active agent than the first 25% of the released active agent from an equivalent dose immediate release (IR) dosage form that releases at least 95% drug in the first 30 minutes.

As described herein, “Saccadic Eye Velocity” (SEV), a psychometric method, can be measured using the following test method: a subject's head is restrained and an light emitting object placed in front of eyes is moved to a certain angle either to left or right in front of eyes and the speed with which the eye ball moves to follow the light is measured as a function of time.

As used herein, the term “pregnancy support” when used to describe the functionality of the oral dosage forms of the present invention, can refer to delaying or preventing the occurrence of undesirable pregnancy conditions from inception through birth including, but not limited to preterm birth, preterm labor, and miscarriage. The pregnancy support can provide improved quality of the pregnancy for the pregnant woman, the fetus, or both. Further, pregnancy support can also include increased fertility for a woman trying to become pregnant.

As used herein, the term “substantially free of” as it refers to the presence or lack of a particular composition or ingredient or component in a given formulation refers to the complete or near complete absence of the ingredient from the formulation such that the ingredient, if present, forms only a minor component or impurity of the formulation. For example, a composition that is substantially free of edible oils may contain a small amount of edible oil impurities that may be present in commercially available surfactants or other commercially available non-edible oil compositions. In one aspect, a formulation that is substantially free of edible oils could have less than 10 wt % of edible oils present in the formulation. In another aspect, a formulation that is substantially free of oils could have less than 5 wt % of edible oils present in the formulation. In yet another aspect, a formulation that is substantially free of edible oils could have less than 2.5 wt % of edible oils present in the formulation.

As used herein, “edible oil” is any oil which can be safely consumed by a mammal. These oils will generally be selected from those oils generally regarded as safe for pharmaceutical or culinary use. Suitable edible oils for the present invention include, but are not limited to, safflower oil, linseed oil, soybean oil, corn oil, sunflower oil, sesame oil, olive oil, cottonseed oil, flaxseed oil, menhaden oil. For the purpose of this invention, the primary characteristic of an “edible oil” is that they are triglycerides of long chain fatty acids with carbon chain length of 12 to 18 and do not include oils which have carbon chain length greater than 20 such as oils containing omega fatty acids, example fish oil, flax seed oil, algae oil and the like.

The terms “release modifying agent”, “release modulating agent”, and “release modifiers” are used interchangeably and refer to pharmaceutically acceptable agents or devices that are able to alter, delay, target, increase or decrease, or otherwise customize, the release rates of at least one of the contents of the compositions or dosage form, when exposed to an aqueous use environment.

By “osmotic agent” is meant any agent that creates a driving force for transport of water from the environment of use into the core of the dosage form.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

Progesterone in serum can be analyzed by specific methods like LC-MS or with not very specific radio immune assay e.g. Advia Centaur® System. The Advia Centaur® progesterone assay is a competitive immunoassay using direct chemi-luminescent technology. Progesterone in the subject sample binds to an acridinium ester-labeled mouse monoclonal anti-progesterone antibody in the “Lite Reagent.” Unbound antibody binds to a progesterone derivative, covalently coupled to paramagnetic particles in the “Solid Phase.” Acid and base reagents initiate the chemi-luminescent reaction. An inverse relationship exists between the amount of progesterone present in the subject's sample and the amount of relative light units (RLU) detected by the system. Subject specimens and all reagents are automatically pipetted by the instrument. Results are calculated off a curve of known concentration of progesterone (calibration curve). Controls of known concentrations are run throughout the assay.

Similarly, pregnane metabolites in serum can be analyzed by specific methods like chromatography or the like. The determination of 5α and 5β pregnanolone can be performed by gas chromatography-mass spectrometry with stable isotope dilution. Briefly known amounts of deuterium labeled analogues are added to plasma samples which are then equilibrated and extracted. The extracts were purified by liquid chromatography using Sephadex LH-20, derivatized and selected ion monitoring is performed at nominal masses m/z 496 and 500, corresponding to the characteristic ions of the heptafluorobutyrates of the native and the labeled pregnanolone, respectively.

It has to be understood that any relative comparisons of blood plasma levels of any compound should be made with the same assay methodology, or corrections must be made to adjust for discrepancy for assay specificity.

As used herein, the term “about” is used to provide flexibility to a numerical range endpoint by providing that a given value may be “a little above” or “a little below” the endpoint. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Invention

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, variants, modifications, and equivalents as may be included within the spirit and scope of the invention as defined by the appended claims.

It has been discovered that progesterone supplementation can prevent preterm birth (PTB) in some high-risk women, but its mechanism of action is not well known. One third of PTB is associated with preterm premature rupture of membranes (PPROM). Without being limited by theory, it has been hypothesized that progesterone is an essential immunomodulatory agent. It plays a critical role in modulation, expression and inhibition of various growth factors, cytokines, cell adhesion molecules and decidual proteins. It may block pro-inflammatory cytokine-induced apoptosis of fetal membrane, thereby preventing PPROM, and PTB. Progesterone inhibits basal and TNF-α-induced apoptosis in term fetal membranes. This may explain in part the mechanism by which progesterone supplementation prevents PPROM and PTB in some high-risk women.

In women, typical progesterone levels are relatively low during the pre-ovulatory phase of the menstrual cycle, rise after ovulation, and are elevated during the luteal phase. Progesterone levels tend to be <2 ng/ml prior to ovulation and >5 ng/ml after ovulation. With the onset of the luteal-placental shift in progesterone support of the pregnancy, levels start to rise further and may reach 100-200 ng/ml at full term. After delivery of the placenta and during lactation, progesterone levels are very low.

During pregnancy, it has been shown that serum progesterone levels are decreased in intrauterine death, premature labor, threatened premature labor, premature rupture of membranes, amnionitis and abruption of placenta. It has been discovered that progesterone has potential for use in pregnancy to treat and or prevent the following conditions or occurrences: spontaneous abortion in women who have had previous spontaneous abortion, history of recurrent spontaneous abortion, previous stillbirth, previous prematurity (<37 weeks), previous premature (<37 weeks) rupture of membranes or PROM, previous pregnancy related hypertension or toxemia, previous abruption of placenta, threatened premature labor or cerclage, multiple pregnancy, primary or secondary infertility, congenital uterine anomaly or any other condition where endogenous progesterone levels are lower than in normal pregnancy.

Primary and secondary outcome measures can be used to determine the need for and/or the effectiveness of progesterone supplementation therapy for a particular subject. Typical primary and secondary outcome measures for preterm birth and preterm labor include, without limitation,

### Primary Outcome Measures (Maternal):

1. Perinatal mortality
2. Preterm birth (less than 32 weeks' gestation)
3. Preterm birth (less than 34 weeks' gestation)
4. Preterm birth (less than 37 weeks' gestation)
5. Major neuro-developmental handicap at childhood follow up

### Secondary Outcome Measures (Maternal):

1. Threatened preterm labor
2. Pre-labor spontaneous rupture of membranes
3. Adverse drug reaction
4. Pregnancy prolongation (interval between randomization and birth)
5. Mode of birth
6. Number of antenatal hospital admissions
7. Satisfaction with the therapy
8. Use of tocolysis

### Secondary Outcome Measures (Infant):

1. Birth before 37 completed weeks
2. Birth before 34 completed weeks
3. Birth before 32 completed weeks
4. Birth before 28 completed weeks
5. Birth weight less than the third centile for gestational age
6. Birth weight less than 2500 grams
7. Apgar score of less than seven at five minutes
8. Respiratory distress syndrome
9. Use of mechanical ventilation
10. Duration of mechanical ventilation
11. Intraventricular hemorrhage—grades III or IV
12. Periventricular leucomalacia
13. Retinopathy of prematurity
14. Retinopathy of prematurity—grades III or IV
15. Chronic lung disease
16. Necrotizing enterocolitis
17. Neonatal sepsis
18. Fetal death
19. Neonatal death
20. Admission to neonatal intensive care unit
21. Neonatal length of hospital stay
22. Teratogenic effects (including virilisation in female infants)

### Secondary Outcome Measures (Child):

1. Major sensorineural disability (defined as any of legal blindness, sensorineural deafness requiring hearing aids, moderate or severe cerebral palsy, or developmental delay or intellectual impairment)
2. Developmental delay
3. Intellectual impairment
4. Motor impairment
5. Visual impairment
6. Blindness
7. Deafness
8. Hearing impairment
9. Cerebral palsy
10. Child behavior
11. Child temperament
12. Learning difficulties
13. Growth assessments at childhood follow up (weight, head circumference, length, skin fold thickness)

### In-Vitro Fertilization

### 1. Primary Outcome Measures:

1.1. Pregnancy Rate
1.2. Live Birth
1.3. Ongoing pregnancy rate
1.4. Clinical pregnancy, defined as ultrasound evidence of fetal heart activity at 6-8 weeks of gestation
1.5. Fetus Vitality measured by heart beat
1.6. Rate of complete abortion 24-48 hrs after receiving medical treatment for early pregnancy failure.

### 2. Secondary Outcome Measures:

2.1. Clinical Pregnancy
2.2. Cycle Cancellation Rates
2.3. Number of Oocytes Generated
2.4. Number of Embryos Generated
2.5. Serum hormonal evaluation
2.6. Follicular Fluid Evaluation
2.7. Peak estradiol level
2.8. Ampules of gonadotropins required during ovarian stimulation
2.9. Number of days of ovarian stimulation
2.10. Number of oocytes retrieved
2.11. Number of embryos transferred
2.12. Number of embryos frozen
2.13. Embryo Grade
2.14. Implantation rate
2.15. Miscarriage Rate
2.16. Pregnancy outcome
2.17. rate of complete abortion at one week, time to expulsion of products of conception, correlation of abortion rates to serum progesterone levels and type of pregnancy failure, number of bleeding days and patient satisfaction
2.18. Ovarian Response [assessed upon completion of the controlled ovarian stimulation and the egg collection procedures]

### Miscarriage

### 1. Primary Outcomes

1.1. Miscarriage
1.2. Early miscarriage up to 12 weeks
1.3. Miscarriage later than 12 weeks and less than 23 weeks
1.4. Cytokine ratio IFN/IL-10
1.5. Clinical pregnancy rate at 8 weeks and 12 weeks of pregnancy
1.6.

### 2. Secondary Outcomes

2.1. Mother
   a. Pain relief (threatened miscarriage)
   b. Severity of ‘morning sickness’-intensified headache
   c. nausea, breast tenderness
   d. reported thromboembolic events
   e. Thrombolytic events
   f. depression;
   g. admission to special care unit
   h. subsequent fertility.
   i. PIBF level
   j. Uterine contraction frequency
2.2. Child
   a. Preterm birth;
   b. stillbirth;
   c. neonatal death;
   d. low birthweight less than 2500 g
   e. fetal genital abnormalities;
   f. teratogenic effects (impairing normal fetal development);
   g. admission to special care unit.
2.3. General
   a. Intrauterine fetal death
   b. Still birth
   c. Fetal
   d. Exploratory analysis of pregnancy outcome by monitoring biochemical and clinical pregnancy parameters, weekly evaluation of serum progesterone
   e. live birth rate, cycle cancellation rate, rate of spontaneous abortion, rate of biochemical pregnancy, rate of ectopic pregnancy

Several biomarkers have been implicated in predicting preterm birth (PTB). Among symptomatic women, the likelihood ratio (LR+) for the prediction of PTB is known to be greater than 10 using amniotic fluid (AF) interleukin-6 (IL-6), AF Ureaplasma urealyticum, as well as a multi-marker consisting of cervical IL-6, cervical IL-8, and cervical length (CL). The LR+ is also known to be between 5 and 10 for serum C-reactive protein (CRP). An LR+ between 2.5 and 5 was recorded for serum corticotropin-releasing hormone (CRH), cervical IL-6, serum relaxin.

In asymptomatic women, AFU urealyticum and a multimarker consisting of five individual markers [fFN, CL, serum alpha-fetoprotein (AFP), serum alkaline phosphatase, and serum granulocyte colony-stimulating factor (G-CSF)] predict PTB with an LR+ greater than 10. The LR+ was between 5 and 10 for serum relaxin and CL. LRs+ recorded for serum alkaline phosphatase, salivary estriol, serum CRH, serum G-CSF, cervical IL-6, AF IL-6, cervical fFN, AFP, and chlamydia all ranged between 2.5 and 5. Finally, an LR+ below 2.5 has been documented for serum ferritin, serum CRP, BV, and cervical ferritin.

Miscarriages and possible miscarriages can be categorized in several ways: A) threatened or possible miscarriage—when any bleeding from the uterus occurs before 20 weeks, but the cervix is closed and the fetus is alive; B) Inevitable abortion or miscarriage (inevitable—meaning it cannot be stopped, particularly if there is bleeding from the uterus and the cervix is opening prior to 20 weeks, but neither the fetus nor placenta have passed out of the woman's body)—the membranes around the fetus may or may not have ruptured (broken); C) Incomplete abortion or miscarriage—when a portion of the fetus or placenta has passed out of the uterus prior to 20 weeks gestation while some of the placenta or fetus remains in the uterus; D) Complete miscarriage—complete expulsion of all the membranes around the fetus and the placenta and the cervix closes prior to 20 weeks; E) Missed abortion or miscarriage—death of the fetus prior to 20 weeks gestation with neither the fetus nor the placenta having been expelled from the uterus; F) Recurrent miscarriage—a woman is said to have recurrent miscarriage after she has already had two or more miscarriages in a row; G) Blighted ovum or an-embryonic gestation—occurs when a gestational sac forms inside the uterus, but no fetus is present after seven weeks.

Threatened miscarriage, as demonstrated by vaginal bleeding with or without abdominal cramps within 26 weeks of conception, is a common complication of pregnancy. It occurs in about 20% of recognized pregnancies. Risk of miscarriage is increased in older women and those with a history of miscarriage.

Low serum levels of progesterone or human chorionic gonadotropin (hCG) are a risk factor for miscarriage. Threatened miscarriage causes considerable stress and anxiety for a pregnant woman. One diagnostic criterion is low serum progesterone, but levels vary widely during early pregnancy and any later decline may be attributed to a dysfunctioning placenta. Nevertheless, luteal support is widely used for the management of threatened miscarriage. First trimester pregnancies show risk of miscarriage with declining serum progesterone levels. Levels of <5 ng/ml were associated with a spontaneous miscarriage in 86% of cases compared with only 8% at levels of 20-25 ng/ml. A threshold value of 14 ng/ml has been reported to differentiate between the viable and non continuing pregnancies. Other maternal serum bio markers such as Tumour marker CA-125, Inhibin A, Anandamide and progesterone induced blocking factor (PIBF) are also good indicators of miscarriage risk.

The complex role of progesterone in pregnancy is becoming increasingly recognized in terms of modulation of the maternal immune response. During normal pregnancy, there is a shift towards a protective T helper (Th)-2 dominated cytokine balance (e.g. interleukin (IL)-4 and IL-10) and away from Th-1 cytokines (e.g. IL-12 and interferon-gamma). The ratio of Th-1 cytokines to Th-2 cytokines such as IFN to IL-10 is used to monitor potential for miscarriage and as a surrogate marker to monitor benefits of progesterone administration to treat or prevent miscarriage. This shift towards Th-2 cytokines is promoted by PIBF, which is synthesized by activated lymphocytes in the presence of progesterone. Other mechanisms by which PIBF prevents inflammatory and thrombotic reactions towards the fetus include an increase of asymmetric non-cytotoxic blocking antibodies and blockade of natural killer (NK) cell degranulation. It is also known that PIBF levels fail to increase in pregnancies that end in miscarriage. Progestogens also have a direct pharmacological effect by reducing the synthesis of prostaglandins, thereby relaxing uterine smooth musculature and preventing inappropriate contractions that may result in miscarriage.

Although the oral dosage forms and methods of the present invention can be used in most female subjects, patients most suitable for receiving oral progesterone of this invention are the ones that have one or more of the following conditions, symptoms, and/or needs: 1) are in need of an anti-inflammatory; 2) are progesterone deficient with base line progesterone in early (first trimester) pregnancy of C_{avg}<14 ng/ml or baseline progesterone levels, C_{avg} of less than 50 ng/ml in late (second and third trimester) pregnancy; 3) have genetic variation of the SERPINH1 gene that cause to produce a reduced amount of the protein, collagen, which may lead to weakened fetal membranes; 4) have a genetic variant of the Prolylcarboxypeptidase gene associated with preeclampsia; 5) have certain bacterial infections (bacterial vaginosis) including Ureaplasma urealyticum, Mycoplasma hominis, Gardnerella vaginalis, and Peptostreptococcus and Bacteroides species; 6) have abnormal amniotic fluid metabolome (the sum of all metabolic processes occurring in the amniotic fluid) indicating risk for prematurity; 7) have had above average total phthalate exposure; 8) abnormal prepregnancy body mass index; 9) have inflammatory milieu of the vagina in early pregnancy; 10) have increased maternal plasma urocortin levels; 11) show increased uterine activity as noted by Home Uterine Activity Monitoring; 12) test positive to salivary estriol levels predicting preterm delivery; 13) show alarming fetal Fibronectin Screening (fFS) results; 14) show unusual cerivical shortening relative to gestational age as measured by cervical ultrasonography, or transvaginal ultrasound or digital examination with/without use of Cervilenz™; 15) show unusual maternal serum bio markers such as Tumour marker CA-125, or Inhibin A, or Anandamide or Progesterone Induced Blocking factor (PIBF); 16) have unbalanced ratio of Th-1 cytokines to Th-2 cytokines such as IFN to IL-10.

Besides maintaining pregnancy, other potential uses of the progesterone containing oral dosage forms of the present invention include, but are not limited to: a) preventing estrogen dominance; b) stimulating new bone formation and prevent/reverse osteoporosis; c) provide the precursor for adrenal cortex hormones (corticosteroids); d) treat variety of skin problems such as acne in adult women, seborrhea, rosacea, psoriasis, and keratoses; e) promote myelin sheath production to protect nerve fibers and speed nerve signals; f) manage depression that accompany PMS, menopause, postpartum depression, etc.; g) protect from brain/spinal cord injury, stroke, and/or hemorrhage.

The present invention provides for progesterone containing pharmaceutical oral dosage forms and related methods. The oral dosage forms can each include an amount of progesterone as well as a pharmaceutically acceptable carrier. The oral dosage forms can be formulated to have at least one of the following characteristics: the oral dosage form produces a pregnane metabolites mean blood plasma level, after administration of single dose of progesterone composition, of less than about 1000 nmol/L; the oral dosage form produces a pregnane metabolites mean blood plasma level, after administration of single dose of progesterone composition, such that the ratio of pregnane metabolite level to parent progesterone level is less than about 10:1; has a dissolution rate in vitro, when measure using a USP Type-1 dissolution apparatus in 900 mL of deionized water with 2.0% (w/v) of sodium lauryl sulfate at 100 rpm, such that the oral dosage form releases at least 10% is released in the first 30 minutes, has a dissolution rate in vitro, when measured using a USP Type-1 dissolution apparatus in 900 mL of deionized water with 2.0% (w/v) of sodium lauryl sulfate at 100 rpm, such that the oral dosage form release less than 45% in the first 4 hours; and the oral dosage form produces a ratio of mean plasma progesterone AUC to an amount of progesterone administered of more than 1.5×10⁻⁶ hr/mL:1.

In another embodiment, the oral dosage form produces a pregnane metabolites mean blood plasma level, after administration of single dose of progesterone composition, such that the ratio of the pregnane metabolite to the parent progesterone level is less than 2.5:1.

In another embodiment, the oral dosage form produces a pregnane metabolites mean blood plasma level, after administration of single dose of progesterone composition, such that the ratio of the pregnane metabolite to parent progesterone level is less than 1:1.

In another embodiment, the oral dosage forms can be formulated to have at least one of the following characteristics: the oral dosage form produces an pregnane metabolites mean blood plasma level, after administration of single dose of progesterone composition, of less than about 500 nmol/L.

In another embodiment, the oral dosage forms can be formulated to have at least one of the following characteristics: the oral dosage form produces an pregnane metabolites mean blood plasma level, after administration of single dose of progesterone composition, of less than about 250 nmol/L.

In a further embodiment, a method of use of the dosage forms of this invention consisting of using less than 200 mg dose of progesterone, given twice or more per day wherein the method produces a pregnane metabolites mean blood plasma level, after administration to a female such that the ratio of pregnane metabolite level to parent progesterone level is less than 10:1.

In a further embodiment, a method of use of the dosage forms of this invention consisting of using less than 200 mg dose of progesterone, given concomitantly with food rich in fat or calories such as standard fat meal wherein the method produces a pregnane metabolites mean blood plasma level, after administration to a female such that the ratio of pregnane metabolite level to parent progesterone level is less than 10:1.

In yet a further embodiment, a method of delaying rise of at least 50 ng/ml of post luteal fetal fibronectin levels in a woman that is at least 16 weeks pregnant. The method includes orally daily administering to the woman at least 50 mg/day of progesterone. It is noteworthy that the oral dosage forms of the present invention can be used as means of administration for this method. Further, the woman in need of the treatment can be asymptomatic or symptomatic of pre-term birth.

The disclosure also includes a method of preventing or reducing dizziness associated with the oral administration of progesterone. The method includes administering any of the oral dosage forms set claimed hereinto a subject. The reduction in dizziness associated with the administration can be quantified or measured by an increase in saccadic eye velocity (SEV) of the subject of at least, as compared to the saccadic eye velocity of the subject after receiving an equivalent dose of progesterone using dosage form containing micronized progesterone suspended in peanut oil and which provides equivalent progesterone AUC values

The oral dosage forms of the present disclosure can be formulated to include from about 10 mg to about 600 mg of progesterone. In another embodiment, the oral dosage form can include about 10 mg to about 400 mg of progesterone. In one embodiment, the oral dosage form can include about 25 mg to about 200 mg of progesterone. In another embodiment, the oral dosage form can include about 25 mg to about 95 mg progesterone. The progesterone can be present in the compositions in any form known in the art. As needed, in the compositions of the present invention, the use of progesterone can be micronized, nano-sized, and/or amorphous forms. In one embodiment, the progesterone can be present or added to the oral dosage form as unmicronized, milled and sieved forms. In another embodiment, the oral dosage form can include a combination of these forms. The progesterone can be solubilized in one or more of the other components of the oral dosage form, such as the carrier, or it can be suspended within the oral dosage form. The suspended portion of progesterone may be partially or completely in unmicronized, milled, sieved, or amorphous forms or combinations thereof.

The progesterone in the compositions of the present invention can be partially or fully in the form of a high-energy solid which increases the dissolution rate in an aqueous medium significantly compared to at least one of its unmilled or unmicronized crystalline forms (low-energy forms). Examples of high-energy forms include amorphous forms and the like. In one embodiment the high-energy form progesterone of present invention may be physico-chemically pure. In yet another embodiment the high-energy form progesterone is physically and/or chemically associated with at least one additional substance, such as for example alcohol, pyrollidone, cellulose, polyol, polyethylene glycol, dextrins, cyclodextrins and the like. Several methods known in the art may be used to produce the high-energy form progesterone of the present invention, for example co-precipitation, solid-solution, co-melting, co-grinding, spray drying with co-solvent, controlled precipitation from super-saturated solutions, solidified super-saturated solutions, and combinations thereof.

Depending on the form of the progesterone, the compositions of the present invention could comprise dissolution-rate enhancers such as for example, wetting agents, surfactants, and the like. In one embodiment the compositions comprise at least one wetting agent and/or surfactant selected from the group comprising hydrophilic, lipophilic, amphiphilic, ionic, non-ionic surfactants. In another embodiment, the composition can be substantially free of added hydrophilic surfactants.

In one embodiment of the present invention the oral dosage form can include oils containing omega fatty acids. Non-limiting examples of oils containing omega fatty acids, can include, but are not limited to, a-linolenic acid (ALA), eicosapentaenoic acid (EPA), and docosahexaenoic acid (DHA), all of which are polyunsaturated with carbon chain length greater than 20. In another embodiment omega-3 fatty acids can be administered with progesterone concomitantly or sequentially.

The oral dosage forms of the present invention can include a pharmaceutically acceptable carrier. The carrier can be a single ingredient, or a mixture of ingredients. Additionally, the carrier can take the form of an encapsulation coat, an absorbing agent, a coating substance, a controlled release device, a release modifying or release controlling agent, surfactants, or a combination thereof. When the carrier includes a surfactant, the surfactant may increase the solubility of the progesterone or other active agent in the system. In some aspects, the carrier can comprise about 1 wt % to about 99 wt % of the total system. In one embodiment, the carrier can comprise about 5 wt % to about 95 wt % of the total system or formulation. In another embodiment, the carrier can comprise about 20 wt % to about 80 wt %. In yet a further embodiment, the carrier can comprise about 30 wt % to about 60 wt %. In one embodiment, the carrier can be admixed with the progesterone. In another embodiment, the carrier can adsorb, entrap, or encapsulate at least a portion of the progesterone. In yet another embodiment, the carrier can act to solubilize the progesterone.

In another embodiment, the carrier and the progesterone may be present separate from each other, but within a unit dosage form. In another embodiment, the carrier and the progesterone may be present as separate unit dosage forms suitable for concomitant or non concomitant oral administration.

Non-limiting examples of compounds that can be used as at least a part of the carrier include without limitation celluloses; dextrins; gums; carbomers; methacrylates; sugars; lactoses; inorganic carbonates, oxides, chlorides sulphate and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

In one embodiment, the carrier can include at least one component selected from celluloses; dextrins; gums; carbomers; methacrylates; inorganic carbonates; salts of calcium; salts of magnesium; fatty acids; fatty acid esters; gelatin; lactoses; polyethylene glycol; polyethylene oxide co-polymers; silicates; partially hydrogenated vegetable oils, fully hydrogenated vegetable oils, waxes, antioxidants, tocopherol, sugar stearates, starches, shellac, resins, proteins, and combinations thereof.

In another embodiment, the carrier can include at least one component selected from celluloses; dextrins; gums; carbomers; methacrylates; sugars; lactoses; inorganic carbonates; salts of calcium; salts of magnesium; salts of fatty acids; inorganic and organic acids; bases and salts; propylene glycol; glycerols; fatty acids; fatty alcohols; fatty acid esters; glycerol esters; mono-, di-glycerol esters of fatty acids; omega oils; waxes; alcohols; gelatin; polyethylene glycol; polyethylene oxide co-polymers; silicates; antioxidants, tocopherol, sugar stearates, starches, shellac, resins, proteins, acrylates; methyl copolymers; polyvinyl alcohol; starch; phthalates; and combinations thereof.

In one embodiment, the oral dosage form can be substantially free of oils having a carbon chain length of 12 to 18 carbons. In another embodiment, the oral dosage form can be substantially free of hydrophilic surfactants. It is important to note that carrier compositions used in the present invention may serve multiple functional purposes within the oral dosage form. For example, a carrier may also function as a disintegrant.

Non-limiting examples of celluloses or cellulosics than can be included in the carrier can include microcrystalline cellulose, ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate (CA), cellulose propionate (CPr), cellulose butyrate (CB), cellulose acetate butyrate (CAB), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC). A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL® series E5, E15LV, E50LV and K100LY, while high viscosity HPMC polymers include E4MCR, E10MCR, K4M, K15M and K100M; especially preferred in this group are the METHOCEL® K series. Other commercially available types of HPMC include the Shin Etsu METOLOSE® 90SH series.

Non-limiting examples of release modifying agents that can be included as the carrier or a component of the carrier can include: polyethylene glycols having a weight average molecular weight of about 1000 and more, carbomer, methyl methacrylate copolymers, methacrylate copolymers, hydroxypropyl methyl cellulose, hydroxypropyl cellulose, cellulose acetate phthalate, ethyl cellulose, methyl cellulose and their derivatives; ion-exchange resin; mono-, di-, tri-esters of fatty acids with glycerol; tocopherol and its esters; sucrose esters with fatty acids; polyvinyl pyrollidone; xanthan gums; cetyl alcohol; waxes; fats and oils, proteins, alginate, polyvinyl polymers, gelatins, organic acids, and their derivatives and combinations thereof.

The dosage form of the present invention may contain different excipients to improve performance, handling, or processing. Generally, excipients such as rate controlling agents, surfactants, pH modifiers, fillers, matrix materials, complexing agents, solubilizers, pigments, Disintegrants, lubricants, glidants, flavorants, inert core agents, and so forth may be used for customary purposes and in typical amounts without adversely affecting the properties of the controlled release dosage form. See for example, Remington's Pharmaceutical Sciences (18th ed. 1990).

Non-limiting examples of fillers, or diluents include celluloses, lactose, mannitol, xylitol, dibasic calcium phosphate (anhydrous and dihydrate) and starch. Non-limiting examples of disintegrants include sodium starch glycolate, sodium alginate, carboxy methyl cellulose sodium, and croscarmellose sodium, and crosslinked forms of polyvinyl pyrrolidone such as those sold under the trade name CROSPOVIDONE (available from BASF Corporation).

Non-limiting examples of binders can include methyl cellulose, microcrystalline cellulose, starch, and gums such as guar gum, and tragacanth. Non-limiting examples of lubricants can include magnesium stearate, calcium stearate, and stearic acid. Non limiting examples of preservatives can include sulfites (an antioxidant), benzalkonium chloride, methyl paraben, propyl paraben, benzyl alcohol and sodium benzoate.

Non-limiting examples of suspending agents or thickeners can include xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, and titanium dioxide. Non-limiting examples of anti-caking agents or fillers include silicon oxide and lactose. Non-limiting examples of solubilizers can include ethanol, propylene glycol or polyethylene glycol.

The addition of pH modifiers such as acids, bases, or buffers may be beneficial, retarding the dissolution of progesterone (e.g., bases such as sodium acetate or amines) or, alternatively, enhancing the rate of dissolution of progesterone (e.g., acids such as citric acid or succinic acid). Other conventional excipients may also be employed in the oral dosage forms of this invention, including those well-known in the art. Generally, excipients such as pigments, lubricants, flavorants, and so forth may be used for customary purposes and in typical amounts without adversely affecting the properties of the compositions.

The dosage form(s) are not limited with respect to size, shape or general configuration, and may be formulated into a variety of dosage forms including, but not limited to two piece hard gelatin capsules, soft gelatin capsules, beads, beadlets, granules, spherules, pellets, microcapsules, microspheres, nanospheres, nanocapsules, tablets, or combinations thereof. Other oral dosage forms known to those of ordinary skill in the art may also be used. In one aspect, the oral dosage form may be a capsule or tablet. In addition, the dosage form may be a drink or beverage solution or a spray solution that is administered orally. Thus, for example, the drink or beverage solution may be formed by adding a therapeutically effective amount of the composition in, for example, a powder or liquid form, to a suitable beverage, e.g., water or juice. In one embodiment, the oral dosage form is a solid oral dosage form.

The progesterone of the oral dosage forms of the present invention may be incorporated into an osmotic sustained or controlled release dosage form. Such dosage forms have at least two components: (a) the core which contains an osmotic agent and progesterone; and (b) a water permeable, non-dissolving and non-eroding coating surrounding the core, the coating controlling the influx of water to the core from an aqueous environment of use so as to cause drug release by extrusion of some or all of the core to the environment of use. The osmotic agent contained in the core of this dosage form may be an aqueous-swellable hydrophilic polymer or it may be an osmogen, also known as an osmagent. The coating can be polymeric, aqueous-permeable, and can have at least one delivery port which is pre-formed or formed in situ. Examples of such dosage forms are well known in the art. See, for example, Remington: The Science and Practice of Pharmacy, 20.sup.th Edition, 2000.

Exemplary osmotic agents that can be used in the oral dosage forms of the present invention include water-swellable hydrophilic polymers, and osmogens (or osmagens). Thus, the core may include water-swellable hydrophilic polymers, both ionic and nonionic, often referred to as “osmopolymers” and “hydrogels.” The amount of water-swellable hydrophilic polymers present in the core may range from about 5 to about 80 wt %, preferably 10 to 50 wt %. Exemplary materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carbox cellulose (CEC), sodium alginate, polycarbophil, gelatin, xanthan gum, and sodium starch glycolate. Other materials include hydrogels comprising interpenetrating networks of polymers that may be formed by addition or by condensation polymerization, the components of which may comprise hydrophilic and hydrophobic monomers such as those just mentioned. Preferred polymers for use as the water-swellable hydrophilic polymers include PEO, PEG, PVP, sodium croscarmellose, HPMC, sodium starch glycolate, polyacrylic acid and crosslinked versions or mixtures thereof.

The oral dosage forms of the present invention can include a coating, such as an enteric coating. One class of preferred coating materials are the pharmaceutically acceptable methacrylic acid copolymer which are copolymers, anionic in character, based on methacrylic acid and methyl methacrylate, for example having a ratio of free carboxyl groups:methyl-esterified carboxyl groups of 1:>3, e.g. around 1:1 or 1:2, and with a mean molecular weight of 135000. Some of these polymers are known and sold as enteric polymers, for example having a solubility in aqueous media at pH 5.5 and above, such as the commercially available EUDRAGIT enteric polymers, such as Eudragit L 30, a cationic polymer synthesized from dimethylaminoethyl methacrylate, Eudragit S and Eudragit NE.

The coating may include conventional plasticizers, including dibutyl phthalate; dibutyl sebacate; diethyl phthalate; dimethyl phthalate; triethyl citrate; benzyl benzoate; butyl and glycol esters of fatty acids; mineral oil; oleic acid; stearic acid; cetyl alcohol; stearyl alcohol; castor oil; corn oil; coconut oil; and camphor oil; and other excipients such as anti-tack agents, glidants, etc. For plasticizers, triethyl citrate, coconut oil and dibutyl sebacate are also useful. Typically the coating may include from about 0.1 to about 25 wt. % plasticizer and from about 0.1 to about 10 wt % anti-tack agents.

The enteric coating may also include insoluble materials, such as shellac, alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin, 1,2-, 3,4-diepoxybutane, etc. The enteric coating may also include starch and/or dextrin.

The coating, including enteric coatings, may be applied to the oral dosage form by dissolving or suspending the enteric coating materials in a suitable solvent. Examples of solvents suitable for use in applying a coating include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, and octane; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; acetonitrile; water; and mixtures thereof.

Coating may be conducted by conventional techniques, such as by pan coaters, rotary granulators and fluidized bed coaters such as top-spray, tangential-spray or bottom-spray (Wurster coating), most preferably the latter. One preferred coating solution consists of about 40 wt % Eudragit L30-D55 and 2.5 wt % triethylcitrate in about 57.5 wt % water. This enteric coating solution may be coated onto the core of the oral dosage form using a pan coater.

The enteric coating materials listed above can be used to granulate a progesterone containing mixture. The resultant granulate may be filled into capsules or compressed to form tablets or caplets.

The release of progesterone from the oral dosage forms or components of the dosage form (e.g. granules), of the present disclosure can be controlled. The oral dosage forms of the present invention can be formulated for once-a-day or twice daily (i.e. once every 12 hours) administration of progesterone.

The oral dosage forms of the present disclosure are able to provide equivalent therapeutic effect to other commercially available dosage forms while at the same time reducing the required daily dosage amounts. One method of demonstrating the increased therapeutic effectiveness of the systems of the present invention is by quantifying their progesterone AUC value to dosage amount ratio as compared to that of commercially available oral progesterone systems having equivalent dosage amounts. By way of example, a 100 mg containing oral dosage form of the present invention provides a higher AUC value as compared to a 100 mg dosage form of micronized progesterone suspended in peanut oil, such as one commercially available in the art. The ability of the present invention to provide this enhanced bioavailability and therefore, improved therapeutic efficacy, is one advantage of the disclosed oral dosage forms.

With the above in mind, the systems of the present invention can produce mean AUC values of progesterone of about 40 ng*hr/ml to about 1800 ng*hr/ml. In another embodiment, the systems can produce mean AUC values of progesterone of about 40 ng*hr/ml to about 1100 ng*hr/ml. The systems of the present invention provide the effect of producing mean progesterone AUC values after a single administration that are significantly higher than the mean AUC value provided by an equivalent dose of a of dosage form having micronized progesterone suspended in peanut oil. In other words, the systems of the present application can provide a mean progesterone AUC value after a single administration that is statistically higher than an equivalent dose of dosage form having micronized progesterone suspended in peanut oil. In one embodiment, the oral dosage form can produce a ratio of mean plasma progesterone AUC to amount of progesterone administered of at least 1.5×10⁻⁶ hr/mL. In another embodiment, the oral dosage form can produce a ratio of mean plasma progesterone AUC to amount of progesterone administered of at least 2.0×10⁻⁶ hr/mL. In yet another embodiment, the oral dosage forms can be formulated to provide a fed to fasted dosing AUC ratio of progesterone greater than about 1.05, wherein the both fed and fasted dosing are single dose administration.

The oral dosage forms of the current invention can be administered in single and multi-dose dosing regimens. In one embodiment, the progesterone compositions of the current invention can provide upon a single dose oral administration of 200 mg fasted state or 50 mg in standard fat fed state or standard calorie fed state, a plasma progesterone mean Cₘₐₓ of about 1 ng/mL to about 175 ng/mL. In one embodiment, the Cₘₐₓ can be about 175 ng/mL or less. In another embodiment, the Cₘₐₓ can be about 150 ng/mL or less. In yet another embodiment, the Cₘₐₓ can be about 85 ng/mL or less. In another embodiment, the Cₘₐₓ is dose proportional for progesterone dose from about 25 mg to at least about 400 mg. In yet another embodiment, the oral dosage forms can be formulated to provide a fed to fasted dosing Cₘₐₓ ratio of progesterone that is from about 1.15 to about 6.0, wherein both fed and fasted dosing are single dose administrations.

Further, the current progesterone compositions can provide upon a single oral administration under fasted state, a plasma progesterone mean Cₘₐₓ that is at least 10% lower compared to the Cₘₐₓ obtained following a fasted state administration of an equivalent progesterone dose of commercially available oral dosage form such as, Prometrium. In one embodiment, the Cₘₐₓ obtained from current compositions is about 15% to about 95% lower; preferably about 35% to about 75% lower; compared to the Cₘₐₓ obtained under similar conditions of dosing with an equivalent progesterone dose of commercially available oral dosage.

Additionally, the current progesterone compositions can be formulated to provide upon a single oral administration, at least 10% higher plasma progesterone mean Cₘₐₓ compared to an equivalent progesterone dose of the commercial oral progesterone dosage form such as Prometrium. In one embodiment, the ratio of the Cₘₐₓ from the current composition to that from the commercial dosage form can be about 1.25 ng/mL or higher; about 2.5 ng/mL or higher; or about 4.0 ng/mL or higher.

Further, the current progesterone compositions can be formulated to provide steady state plasma progesterone C_{avg} to females requiring pregnancy support. In one embodiment, the steady state progesterone C_{avg} can be from about 1 ng/mL to about 300 ng/mL, about 200 ng/mL or less, about 150 ng/mL or less, or about 85 ng/mL or less. In a specific embodiment, the steady state progesterone C_{avg} can be less than about 50 ng/mL, specially in early pregnancy, i.e. first 24 weeks. In another embodiment, the steady state progesterone AUC can be about 40 ng*h/mL to about 1800 ng*h/ml; preferably from about 40 ng*h/mL to about 1100 ng*h/mL. In another embodiment, the steady state progesterone C_{avg} or AUC, or both, is dose proportional for progesterone dose from about 25 mg to at least about 400 mg.

In one embodiment, the oral dosage form can be formulated to provide an increase from endogenous base line in Cₘₐₓ of progesterone of at least 11 ng/ml after a single administration to a female requiring pregnancy support in the first trimester of pregnancy. In another embodiment, the oral dosage form can be formulated to provide an increase from endogenous base line in Cₘₐₓ of progesterone of at least 25 ng/ml after a single administration to a female requiring pregnancy support in the second trimester of pregnancy. In yet a further embodiment, the oral dosage form can be formulated to provide an increase from endogenous base line in Cₘₐₓ of progesterone of at least 50 ng/ml after a single administration to a female requiring pregnancy support in the third trimester of pregnancy.

Further, the oral dosage forms of the present disclosure can be used for regular daily oral administration to female requiring pregnancy supports. In one embodiment, the oral dosage form can be formulated such that regular daily administration of the oral dosage form to a female requiring pregnancy support during the first trimester of pregnancy produces an increase from endogenous base line in the steady state C_{avg} of progesterone of at least 11 ng/ml. In another embodiment, the oral dosage form can be formulated such that regular daily administration of the oral dosage form to a female requiring pregnancy support during the second trimester of pregnancy produces an increase from endogenous base line in the steady state C_{avg} of progesterone of at least 25 ng/ml. In yet a further embodiment, the oral dosage form can be formulated such that regular daily administration of the oral dosage form to a female requiring pregnancy support during the third trimester of pregnancy produces an increase from endogenous base line in the steady state C_{avg} of progesterone of at least 50 ng/ml.

The oral dosage forms of the present disclosure can be formulated to provide dissolution rates that yield enhanced therapeutic effect and lengthened therapeutic durations. In one embodiment of the present invention, the oral dosage form can be formulated to have a dissolution rate in vitro, (when measured using a USP Type-1 dissolution apparatus in 900 mL of deionized water with 2.0% (w/v) of sodium lauryl sulfate at 100 rpm), that releases at least 10 wt % of the progesterone in the first 30 minutes following administration. In another embodiment, the oral dosage can provide less a release of less than 45 wt % of the progesterone 4 hours after administration. In yet a further embodiment, the oral dosage form can release at least about 80 wt % of the progesterone about 8 hours after administration.

As discussed above, preterm birth is common complication of pregnancy. Typically, preterm birth is defined as delivery of baby with gestational age less than 37 weeks. In the US, there is one preterm birth per minute and has great unmet need for approved treatment options. Fetal fibronectin (fFN) is a type of fibronectin protein produced by fetal cells and is found at the interface of the charion and the deciduas (between the fetal sac and the uterine lining). During early pregnancy, fetal fibronectin serves as a glue holding the amniotic sac attached to the uterine wall. It is present in vaginal and cervical fluid during the first trimester of pregnancy up to about 22 weeks, then is absent between 22 weeks and 35 weeks of pregnancy, and reappears during the last trimester of pregnancy. The presence of fetal fibronectin in vaginal and cervical secretions can be an indication that preterm labor or birth is going to occur, especially in women at high risk for preterm birth.

Fetal fibronectin may be a good predictor of spontaneous preterm birth before cervical dilation. The fFN diagnostic test is performed by collecting specimen from the patient using a vaginal swab. Special precautions must be taken to avoid a false positive fetal fibronectin result as it can occur if the test is performed after digital examination of the cervix or after having had intercourse. The test may be run on patients between 22 and 35 weeks gestation. From weeks 22 to 35 in pregnancy, there should be very little fFN detectable. fFN can often be detected before other symptoms of preterm labor, such as contractions and changes in cervical length. Fetal fibronectin levels reach their peak of approximately 4000 ng/mL between 10 and 12 weeks gestation, fall to below 50 ng/mL by 18 weeks, and remain at undetectable levels until 36 to 37 weeks. Mechanical stress caused by uterine contractions and local inflammation lead to separation of the chorio-decidival interface and release of fetal fibronectin into the vagina. Between 20 and 37 weeks gestation, fetal fibronectin should normally not be present in the cervix and vagina. Detection of fetal fibronectin in cervico-vaginal secretions at a concentration of >50 ng/mL indicates the patient is at high risk for preterm labor and subsequent early birth.

As described above, it has been discovered that the rise in fetal fibronectin levels in a woman requiring pregnancy support can be suppressed or delayed in women requiring pregnancy support through the administration of oral progesterone in the oral dosage forms of the present invention. In particular, the oral dosage forms of the present disclosure can be used to delay the rise of 50 ng/ml of fetal fibronectin in asymptomatic women that are at least 16 weeks pregnant. The method includes orally administering to the females requiring pregnancy support at least 50 mg/day of progesterone on a daily basis. In one embodiment, the oral administration of the progesterone in the oral dosage form can delay the rise of the fetal fibronectin for at least one week as compared to no or substantially no progesterone treatment. In another embodiment, the oral administration of the progesterone can keep or maintain the fetal fibronectin level in the asymptomatic females requiring pregnancy support below about 200 ng/ml. In a further embodiment, the oral administration of the progesterone can maintain the fetal fibronectin level in the asymptomatic females requiring pregnancy support below about 50 ng/ml.

Further, as described above, the present disclosure provides a method of reducing dizziness or sedation or both associated with the oral administration of progesterone comprising, administering an oral dosage form of this invention to a subject. The reduction in dizziness or sedation or both can be measured as compared to a dosage form containing micronized progesterone suspended in peanut oil and which provides equivalent progesterone AUC values.

The reduction in dizziness associated with the administration can be measured by any known method in the art including the measurement of saccadic eye velocity (SEV) of the subject. Saccadic Eye Velocity (SEV) is one method of measuring extent of dizziness. When progesterone is administered to a human subject orally, pregnane metabolite levels rise due to metabolism and lead to dizziness. A decrease in SEV can indicate that the subject is dizzy or sedated. When a subject is dizzy the saccadic eye velocity tends to be slower.

The present invention includes methods of treating and preventing infertility and miscarriage by providing oral progesterone once or twice daily for at least 6 weeks after becoming pregnant. In one embodiment, a method for management of pre-term labor, maintenance of tocolysis, latency to birth or chronic tocolysis through oral progesterone supplementation until the delivery as the earliest sign of labor in symptomatic women as noted by premature uterine contractions, shortened cervix<3 cm, rise in vaginal fetal fibronectin levels of 50 ng/ml or cervical ripening and significant dilation.

The present invention also provides a method for prevention of pre term birth in patients with high risk pregnancies that include prior history of pre term birth, shortened cervix. In some embodiments, such a method may include orally administering an oral dosage form disclosed herein once or twice daily to a women who is a least 16 weeks pregnant. Take once or twice daily starting as early as week 16 of pregnancy till delivery. Such method can include stepped up dose as the pregnancy progresses, such as 25% dose increase every 4 weeks till delivery.

In one embodiment, the compositions of the current invention can preferentially limit or reduces pre-systemic inactivation of progesterone, especially in the gastro-intestinal lumen and/or during transit through the intestinal wall relative to liver first pass inactivation. In another embodiment, the degradation of progesterone can be limited by releasing substantial amount of the progesterone dose in the post-duodenal region of the intestine. In a particular embodiment, the amount of progesterone dose released in the post-duodenal region of the intestine is about 30% or more; preferably, from about 50% to about 100%, more preferably from about 70% to about 100%. In another embodiment, the dosage form suitable for post-duodenal release of progesterone comprises at least one pH sensitive pharmaceutically acceptable additive that imparts delay release characteristics to the composition. In yet another embodiment, the dosage form is an enteric-coated.

In a further embodiment, the compositions of the present invention can sufficiently limit degradation of progesterone by limiting the degradation enzymes in the gastro-intestinal tract and/or lumen. In one specific embodiment, the composition suitable for limiting the degradation enzymes comprises at least one immediate release progesterone dose fraction and at least one modified release progesterone dose fraction. In a specific embodiment, the immediate release dose fraction can constitute from about 10% to about 90% of the total dose. In another specific embodiment, the modified release dose fraction can constitute from about 10% to about 90% of the total dose.

The present invention also provides for kits used in disbursement and administration of the oral dosage formulations of the present invention. In some aspects, such a kit may comprise the oral dosage form of the present invention along with one or more other components, including, but not limited to 1) instructions to enable those ordinarily skilled in the art to prepare a dosage form for immediate dispensing to the subject in need of; 2) one or more containers filled with one or more of the ingredients of the oral pharmaceutical dosage forms of the invention. Suitable containers include, for example, a bottle, a box, a blister card, a foil packet, or a combination thereof; 3) a tamper proof container or packaging; 4) other pharmaceutical dosage forms including other active agents; 5) Notice or printed instructions: in a form prescribed by a governmental agency regulating the manufacture, use, or sale of pharmaceuticals or biological products, which notice reflects approval by the agency of the manufacture, use, or sale for human administration to treat a condition that could be treated by oral progesterone therapy; 6) A “planner” for monitoring and tracking administration of the oral dosage forms; 7) Containers for storing and transporting the components of the kit. 8) Pregnancy test kits; 9) fetal fibronectin testing kits; 10) progesterone testing materials; 11) tests for identifying patients with high risk of pre term birth; 12) tests for identifying threatened miscarriage and/or pre term labor; 13) vitamins and/or nutritional supplements such as folates, omega fatty acids; 14) utermine monitoring materials; 15) Bacterial infection materials; 16) testing materials for identifying maternal serum protein or non protein biomarker that predicts prematurity in symptomatic or asymptomatic woman; 17) testing materials for amniotic fluid metablome; 18) genetic testing materials for SERPINH1 or Polycarboxy peptidase kit; 19) testing materials for maternal plasma urocortine test; 20) materials to perform cerclage; 21) materials to test for serum markers such as Tumour marker CA-125, or Inhibin A, or Anandamide or Progesterone Induced Blocking factor (PIBF); and 22) materials to measure unbalance in the patient ratio of Th-1 cytokines to Th-2 cytokines such as IFN to IL-10. 23) Pre-recorded media device, 24) testing materials for identifying amniotic/fetal serum protein or non protein biomarker that predicts prematurity in symptomatic or asymptomatic woman, 25) materials to measure cervical length.

The composition methods of this invention are intended, in one aspect, for use in prevention or reduction of vaginal bleeding or management of abdominal pain or management of uterine contractions or sustain fetus viability or improve immunological functions such as The-1-to-The-2 cytokine level ratios, in symptomatic or asymptomatic pregnant female.

### EXAMPLES

The following examples are provided to promote a more clear understanding of certain embodiments of the present invention, and are in no way meant as a limitation thereon. The compositions may be suitably modified by a person skilled in the art to get dosage forms such as capsule, tablet, mould, beads, granules and the like.

### Example 1

### Progesterone Containing Oral Formulations for Oral Delivery

Several progesterone containing formulations are prepared as set forth in Tables I-III. Specifically, formulations 1, 2, 3, 5-1, 7, 9, 10, 10-1, 10-2, 10-3, and 21 are suspension formulations in semi-solid or solid form and are prepared by weighing all the excipients, heating the excipients all together to about 40-to about 77° C. and then cooling the mixture to about 35-65° C. The progesterone is then weighed and added to the excipient mixture and the entire combination is mixed to form a homogenous suspension and then filled into hard gelatin capsules. These formulations when dosed under standard fat fed condition are expected to have Cₘₐₓ and AUC comparable to commercial suspension product at one-half to one-fourth dose of the corresponding commercial suspension product. For example, 200 mg of commercial micronized progesterone suspended in peanut oil (Prometrium®) dosed as recommended should be equivalent to 50 to 85 mg of formulations 1, 2, 3, 5-1, 7, 9, 10, 10-1, 10-2, 10-3, and 21 when dosed with high fat high calorie food. Formulations 5, 6, 8, and 13-22 are progesterone containing solution formulations in liquid or solidified form, and are prepared by weighing all the excipients together and then heating the excipients to about 40-77° C.). The required amount of progesterone is weighed and added to the melted mixture and thoroughly mixed to allow the progesterone to dissolve and form a solution. The progesterone containing solution is then filled into hard gelatin capsules.

Formulations 4, 4-1, 11 to 12-02 and 23 to 37 are each tablet formulations and are prepared by weighing and dry blending all of the formulation excipients and the progesterone. The powder mixture is then compressed into matrix or multi-layer tablets having the required dosage amount. Formulations 12, 26 and 28 are enteric coated tablets and are prepared by weighing and dry-blending the formulation excipients in dry form (except the enteric polymer) with the progesterone. The mixture is then compressed into tablets containing the required dosage amount of progesterone and the tablets are enteric coated using the enteric polymer and well known coating techniques. It is noteworthy that tableting aids and coating aids known in the art can be used in the tableting and/or coating of these Formulations.

TABLE I

| | Formulation No. | | | | | | | | | |
|---|---|---|---|---|---|---|---|---|---|---|
| | 1 | 2 | 3 | 4* | 4-1 | 5 | | 6 | 7 | 8 |
| INGREDIENT** | mg | mg | mg | mg | mg | mg | 5-1 | mg | mg | mg |
| Progesterone*** | 100 | 50 | 100 | 200 | 25 | 25 | 25 | 25 | 25 | 50 |
| Edible Oil**** | 200 | — | 200 | — | — | — | — | — | — | — |
| (e.g. Corn oil) | | | | | | | | | | |
| Omega-3 | — | — | — | — | — | — | — | — | 500 | — |
| Tocopherol | — | — | — | — | — | 150 | 10 | — | — | — |
| Capmul MCM | — | 10 | — | — | — | — | — | 400 | — | 200 |
| Hydrophilic Solidifying | — | — | — | — | — | — | — | — | — | 25 |
| Agent (e.g. PEG 8000) | | | | | | | | | | |
| Lipophilic Surfactant | — | — | 10 | — | — | — | — | — | — | 10 |
| (e.g. Labrafil 2125 CS) | | | | | | | | | | |
| Lipophilic Additive | — | — | 20 | — | — | — | — | — | — | — |
| (e.g. Hydrogenated | | | | | | | | | | |
| Castor Oil) | | | | | | | | | | |
| Hydrophilic Surfactant | 150 | 250 | 100 | — | — | 150 | 150 | 200 | 100 | 80 |
| (e.g Cremophor RH 40) | | | | | | | | | | |
| Hydrophilic Surfactant | — | — | — | 5 | 50 | — | — | — | — | — |
| (e.g. Sodium Lauryl Sulfate) | | | | | | | | | | |
| Diluents (e.g. lactose) | — | — | — | 250 | 250 | — | — | — | — | — |
| Total | 450 | 300 | 430 | 400 | 325 | 325 | 185 | 625 | 625 | 370 |

*Additional Tableting known in the art can be used
**Excipients shown are exemplary of classes of excipients that can be used
***The form of the drug can be interchanged with other forms such as micronized, sieved, milled, amorphous, nano, etc.
****Edible oils refer to oil containing triglycerides of fatty acids

TABLE II

| | Formulation No. | | | | | | | | |
|---|---|---|---|---|---|---|---|---|---|
| | 9 | 10 | 10-1 | 10-2 | 10-3 | 11 | 12 | 12-01 | 12-02 |
| INGREDIENT | mg | mg | mg | mg | mg | mg | mg | mg | mg |
| Progesterone | 200 | 200 | 200 | 200 | 200 | 200 | 200 | 200 | 200 |
| Edible Oil **** | 400 | 350 | 365 | 365 | 365 | — | — | — | — |
| (e.g. Corn oil) | | | | | | | | | |
| Hydrophilic Solidifying | — | 40 | 39 | 39 | 39 | — | — | — | — |
| Agent (e.g. PEG 8000) | | | | | | | | | |
| Hydrophilic Surfactant | — | 10 | 6 | 6 | 6 | — | — | — | — |
| (e.g. Cremophor RH 40) | | | | | | | | | |
| Hydrophilic Surfactant | | | | | | | | 5 | 5 |
| (e.g. Polysorbate 80) | | | | | | | | | |
| Hydrophilic Surfactant | | | | | | | | 50 | 50 |
| (e.g. Sodium Lauryl | | | | | | | | | |
| Sulfate) | | | | | | | | | |
| Lipophilic Solidifying | — | — | — | 12.4 | 39 | — | — | — | — |
| Agent (e.g. Glyceryl | | | | | | | | | |
| Distearate) | | | | | | | | | |
| Hydrophilic Polymer | — | — | — | — | — | 200 | 5 | | |
| (e.g. HPMC) | | | | | | | | | |
| Enteric Polymer | — | — | — | — | — | — | 60 | | 25 |
| (e.g. Eudragits) | | | | | | | | | |
| Diluents/Processing Aids | — | — | — | — | — | 400 | 300 | 245 | 245 |
| Total | 600 | 600 | 610 | 622.4 | 649 | 800 | 565 | 500 | 525 |

* Additional tableting known in the art can be used
** Excipients shown are exemplary of classes of excipients that can be used
*** The form of the drug can be interchanged with other forms such as micronized, sieved, milled, amorphous, nano, etc.
**** Edible oils refer to oil containing triglycerides of fatty acids

TABLE III

| | Formulation No. | | | | | | | | | | | |
|---|---|---|---|---|---|---|---|---|---|---|---|---|
| | 13 | 14-1 | 14-4 | 15 | 16 | 16-1 | 17 | 18 | 19 | 20 | 21 | 22 |
| INGREDIENT | mg | mg | mg | mg | mg | mg | mg | mg | mg | mg | mg | mg |
| Progesterone | 100 | 50 | 50 | 50 | 50 | 50 | 50 | 50 | 50 | 50 | 50 | 50 |
| Medium Chain | | | | | | | | | | | 200 | |
| Triglyceride | | | | | | | | | | | | |
| Tocopherol | 400 | 185 | 185 | 200 | 200 | 185 | | | | | | |
| Capmul MCM | | | | | | | 672 | 650 | 651 | 600 | | 450 |
| Hydrophilic | | 23 | 23 | 25 | 25 | 25 | 50 | | | 50 | 25 | |
| Solidifying Agent | | | | | | | | | | | | |
| (e.g. Polyethylene | | | | | | | | | | | | |
| Glycol 8000) | | | | | | | | | | | | |
| Hydrophilic | | | | | | 40 | | | | | | |
| Solidifying Agent | | | | | | | | | | | | |
| (e.g. Polyethylene | | | | | | | | | | | | |
| Glycol 20000) | | | | | | | | | | | | |
| Lipophilic | | | | | | | 42 | | | 80 | | |
| Solidifying Agent | | | | | | | | | | | | |
| (e.g. Glyceryl | | | | | | | | | | | | |
| Distearate) | | | | | | | | | | | | |
| Lipophilic | | | | | | | | | | | | 150 |
| Solidifying Agent | | | | | | | | | | | | |
| (e.g. Stearic Acid) | | | | | | | | | | | | |
| Lipophilic | | | | | | | | 60 | 60 | | | 40 |
| Solidifying Agent | | | | | | | | | | | | |
| (e.g. Hydrogenated | | | | | | | | | | | | |
| Castor Oil) | | | | | | | | | | | | |
| Hydrophilic | 100 | 77 | 77 | 110 | 150 | 77 | 9 | 64.9 | 9 | 30 | 110 | |
| Surfactant | | | | | | | | | | | | |
| (e.g. Cremophor | | | | | | | | | | | | |
| RH 40) | | | | | | | | | | | | |
| Lipophilic | | | | | | | 9 | 9 | 9 | 10 | | |
| Surfactant Labrafil | | | | | | | | | | | | |
| M 2125 CS | | | | | | | | | | | | |
| Hydrophobic | | | | | | | | 20 | 45 | 40 | | 40 |
| Polymer | | | | | | | | | | | | |
| (e.g. Ethyl | | | | | | | | | | | | |
| Cellulose) | | | | | | | | | | | | |
| Ethyl Alcohol | | 38 | | | | 38 | | | | | | |
| Total | 600 | 373 | | 385 | 425 | | 832 | 855 | 825 | 500 | 385 | 730 |

* Additional tableting known in the art can be used
** Excipients shown are exemplary of classes of excipients that can be used
*** The form of the drug can be interchanged with other forms such as micronized, sieved, milled, amorphous, nano, etc.
**** Edible oils refer to oil containing triglycerides of fatty acids

TABLE IV

| | Formulation | | | | | |
|---|---|---|---|---|---|---|
| | 23 | 24 | 25 | 26 | 27 | 28 |
| INGREDIENT | mg | mg | mg | mg | mg | mg |
| Progesterone | 25 | 25 | 25 | 25 | 25 | 25 |
| Hydrophilic | 2.5 | 2.5 | 2.5 | 2.5 | 2.5 | 2.5 |
| Surfactant | | | | | | |
| (e.g. Tween80) | | | | | | |
| Hydrophilic | 12.5 | 12.5 | 12.5 | 12.5 | 12.5 | 12.5 |
| Surfactant | | | | | | |
| (e.g. Sodium Lauryl | | | | | | |
| Sulfate) | | | | | | |
| Hydrophilic Polymer | | 50 | 100 | | | 100 |
| (e.g. HPMC) | | | | | | |
| Enteric Polymer | | | | 15 | | 15 |
| (e.g. Eudragit) | | | | | | |
| Hydrophobic | | | | | 15 | |
| Polymer (e.g. | | | | | | |
| Ethyl Cellulose) | | | | | | |
| Diluents/Processing | 245 | 245 | 245 | 245 | 245 | 245 |
| Aids | | | | | | |
| Total | 285 | 335 | 385 | 300 | 300 | 400 |

TABLE V

| | Dosage Form | | | | | | | | |
|---|---|---|---|---|---|---|---|---|---|
| | 1 | 2 | 3 | 4 | 5 | 6 | 7 | 8 | 9 |
| % Formulation 23 | 100 | 50 | 50 | 50 | 30 | | | 30 | 50 |
| % Formulation 24 | | 50 | | | | | | | |
| % Formulation 25 | | | 50 | | | 100 | | 30 | |
| % Formulation 26 | | | | 50 | | | | 40 | |
| % Formulation 27 | | | | | | | 100 | | 50 |
| % Formulation 28 | | | | | 70 | | | | |
| Total | 100 | 100 | 100 | 100 | 100 | 100 | 100 | 100 | 100 |

* Additional tableting known in me art can be used
** Excipients shown are exemplary of classes of excipients that can be used
*** The form of the drug can be interchanged with other forms such as micronized, sieved, milled, amorphous, nano, etc.
**** Edible oils refer to oil containing triglycerides of fatty acids
The above dosage forms can be single or multiple formulation units in a capsule or as a single or multiple formulation units as single tablet or multi-layer tablets

### Example 2

### In Vitro Dissolution of Progesterone Containing Compositions

To carry out in-vitro dissolution of the dosage forms of the invention, a dosage form according to the present invention is placed into a stirred USP type 1 dissolution flask containing 900 mL of dissolution medium comprised of DI Water dissolved with 2% w/v sodium lauryl sulfate. In case of enteric coated dosage form relevant dissolution conditions known in the art to be employed. In the flasks, the dosage form is placed in a basket, so that all surfaces are exposed to the moving dissolution media and the solutions are stirred using paddles at a rate of 100 rpm. Samples of the dissolution medium are taken at periodic intervals using auto sampling system. The concentration of dissolved drug in the dissolution medium is then determined by HPLC at a UV absorbance of 245 nm using a UV-Vis detector. Drug concentration is calculated by comparing UV absorbance of samples to the absorbance of drug standards. The mass of dissolved drug in the dissolution medium is then calculated from the concentration of drug in the medium and the volume of the medium, and expressed as a percentage of the mass of drug originally present in the dosage form. Formulations and dosage forms of the invention were tested in accordance to example 2 and the data is presented in figures as described below.

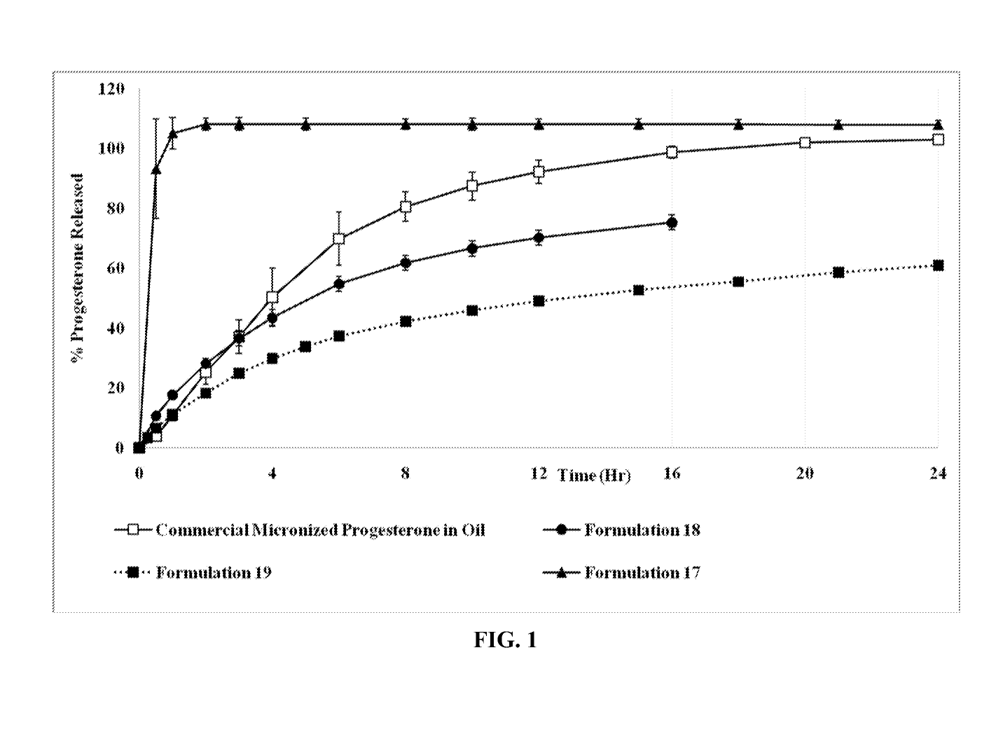

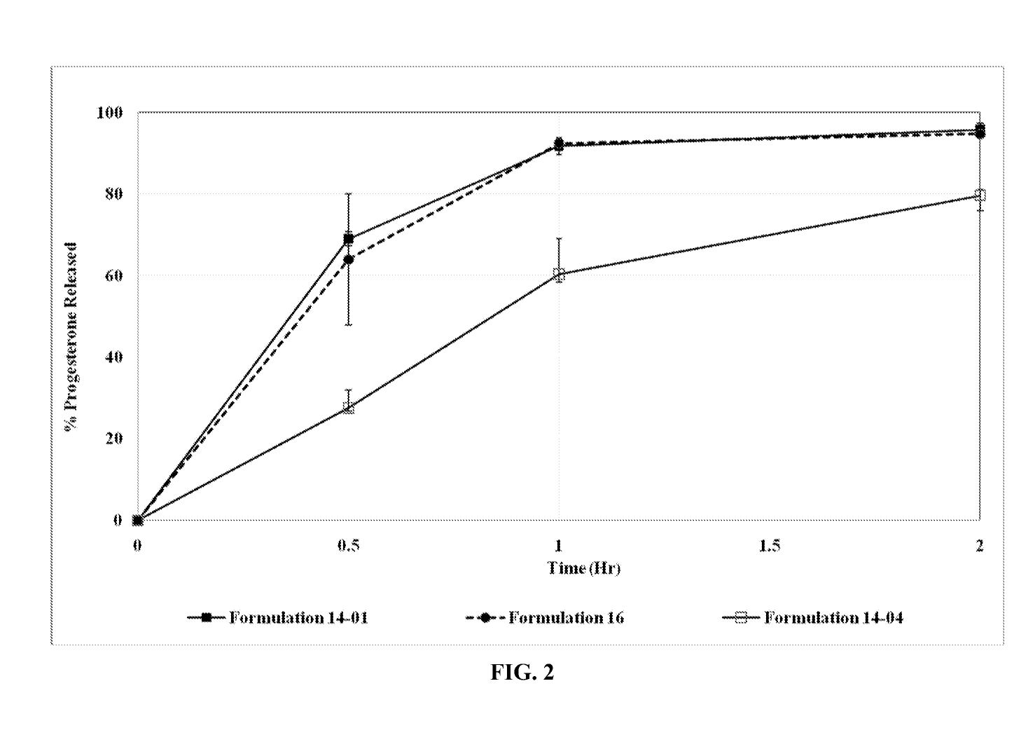

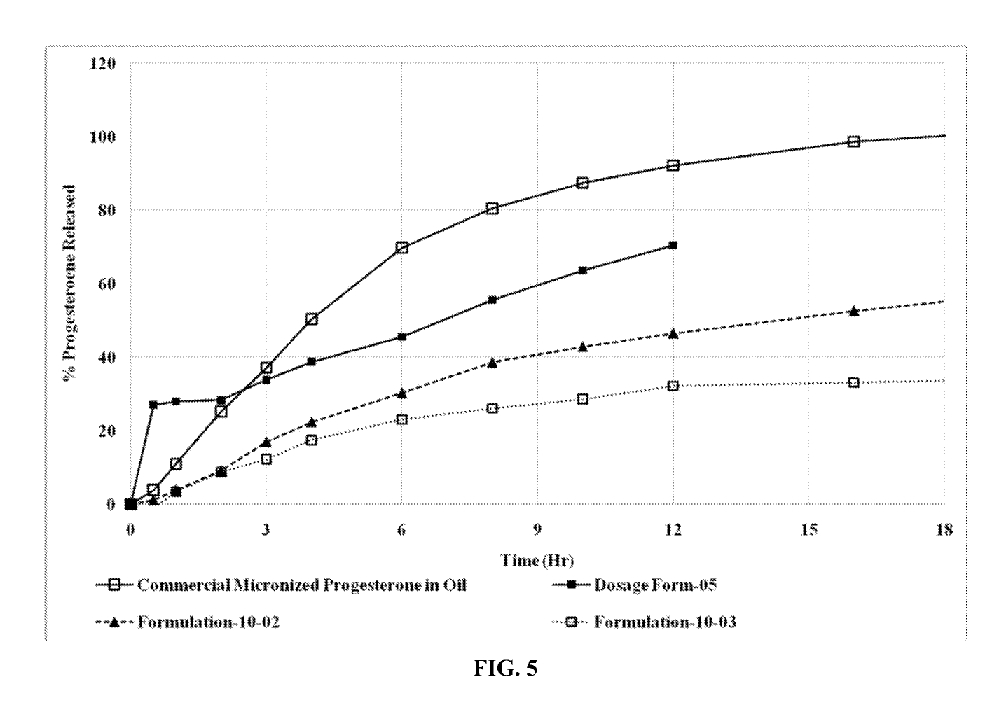

FIG. 1 shows a plot of the release of several of the example formulations. As is shown in FIG. 1, Formulations 18 and 19, compared to Formulations 17, demonstrate how the amount of lipophilic solidifying agent and hydrophobic polymer can affect the release profile of the compositions. Similarly, FIG. 2 shows the amount of the hydrophilic surfactant can affect the release profile of formulations 14-01, 14-04 and 16. FIG. 5 shows the release profiles of a progesterone dosage forms and formulations as compared to commercial micronized progesterone in peanut oil product tested in accordance to Example 2.

### Example 3

### Pharmacokinetic Testing of Progesterone Containing Oral Dosage Forms

Dosage forms of the present disclosure are administered to subjects in a randomized, crossover study. The study is an open-label, randomized, single-dose, crossover study performed on 16 healthy volunteers. A total of 16 subjects complete the clinical phase of the study. In each period, subjects are housed from at least 20 hours before dosing until after the 24-hour blood draw. There is a 7-day washout between each dosing period, during the study; the subjects are monitored for side effects like dizziness.

The Cₘₐₓ, Tₘₐₓ, AUC₀₋ₜ and AUC_{0-α} are calculated for progesterone in the plasma of the test subjects. Pharmacokinetic and statistical analyses are performed on the data obtained from the subjects. This data, in part, is contained in the following tables. The pharmacokinetic parameters are defined as follows:
AUCₒ₋ₜ: The area under the plasma concentration versus time curve, from time 0 to the last measurable concentration of the administered drug, as calculated by the linear trapezoidal method.
AUC (AUC_{0-α}): The area under the plasma concentration versus time curve from time 0 to infinity. AUC was calculated as the sum of the AUC₀₋ₜ plus the ratio of the last measurable plasma concentration of the administered drug to the elimination rate constant.
Cₘₐₓ: The maximum measured plasma concentration of the administered drug.
Tₘₐₓ: The time at which the maximum measured plasma concentration of the administered drug is achieved
C_{avg}: The average plasma concentration of the analyte at steady state.
Mean: Average value of measured parameter of all individual subjects.

Table IV shows the comparative results for administration of the capsules of Formulation 14 in order to demonstrate the correlation between Cₘₐₓ and the incidence of dizziness. Formulation 14-01 refers to the administration of a single capsule of Formulation 14 for a total progesterone dosing amount of 50 mg. Example 14-02 refers to the simultaneous administration of two capsules of Formulation 14 for a total progesterone dosing amount of 100 mg. Similarly, Formulation 14-03 refers to the simultaneous administration of four capsules of Example 14 for a total progesterone dosing amount of 200 mg. The Cₘₐₓ and reported incidence rate of dizziness was recorded and is shown in Table VI.

TABLE VI

| | Total | Cₘₐₓ | Incidence of |
|---|---|---|---|
| Treatment | Dose | (ng/mL) | Dizziness (%) |
| Formulation 14-01 | 50 mg | 57.57 ± 43.14 | 0 |
| (single capsule of | | | |
| Formulation 14) | | | |
| Formulation 14-02 | 100 mg | 125.36 ± 104.85 | 0 |
| (2 capsules of | | | |
| Formulation 14) | | | |
| Formulation 14-03 | 200 mg | 177.30 ± 102.55 | 13.3% |
| (4 capsules of | | | |
| Formulation 14) | | | |

As is shown in Table VI, the Cₘₐₓ value can be directly related to the incidence of dizziness.

Testing is also performed to compare the commercially available progesterone oral suspension product (Prometrium®—Solvay Pharmaceuticals) to Formulation 14-03 (4 capsules of Formulations 14). The administration profile for the testing is set forth in Table VII.

TABLE VII

| Example | Administration | Total Dose |
|---|---|---|
| 14-03 | 4 capsule of 50 mg of dosage form given in | 200 mg |
| | Formulation 14 | |
| | 2 capsules of Commercial suspension 100 mg | 200 mg |

The dosage forms are administered to the test subjects and the Cₘₐₓ, Tₘₐₓ, AUC₀₋ₜ and AUG_{0-α} is calculated for progesterone in plasma. The comparative results of the testing are shown below in Table VIII.

TABLE VIII

| PK | | Formulation | Commercial | % of test to |
|---|---|---|---|---|
| Parameter | Units | 14-03 | Suspension | reference |
| Cₘₐₓ | ng/mL | 177 ± 102 | 59 ± 40 | 354% |
| AUC_{0-α}: | ng * h/mL | 693 ± 420 | 456 ± 318 | 152% |
| Tₘₐₓ | h | 1.5 | 2.0 | 75% |
| AUC/mg | (ng * h/mL)/mg | 3.47 ± 2.1 | 2.28 ± 1.59 | |

### Example 4

### Illustration of Food Effect with Progesterone Formulations

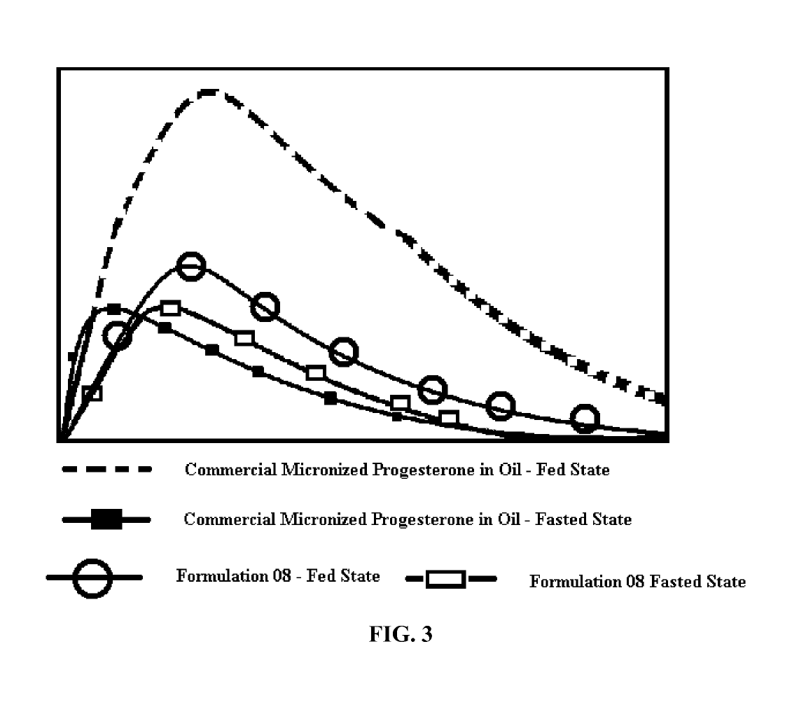

In order to demonstrate the reduced food effect of the progesterone formulations of the present disclosure, comparative measurements are made between the formulation of Formulation 6 and the progesterone oil suspension (Prometrium®) that is commercially available. Subjects receive doses of either the formulation of Formulation 6 or the progesterone oil suspension in both the fed (standard fat/calories meal) and the fasted state and progesterone blood plasma levels are measured. FIG. 3 shows a plot of the progesterone blood plasma levels for each formulation in both the fed and fasted states. As shown in FIG. 3, the formulations of the present disclosure have a significantly reduced food effect as compared to the commerically available oil suspension.

### Example 5

### Administration of Progesterone in Women Requiring Pregnancy Support

Dosage forms of the present disclosure are administered to subjects in a randomized, crossover study. The subjects are women requiring pregnancy support each of whom is at least 16 weeks pregnant. The study is an open-label, multiple-dose study. Prior to administration of the test dosage form, blood samples are collected and measured for plasma progesterone level using a suitable highly sensitive LC-MS or radio immune assay method.

Subjects are dosed with the test dosage form and blood is drawn at regular predetermined time points for a duration of 24 hours and again at 7 days. The collected blood samples are analyzed for progesterone level in the blood. The Cₘₐₓ, C_{avg}, Tₘₐₓ, AUC₀₋ₜ and AUC_{0-α} are calculated for progesterone in plasma. Pharmacokinetic and statistical analyses are performed on the data obtained from the subjects. The dosing regimens are generally described below.
(1) Early pregnancy (First Trimester): (primarily used for luteal phase defect, infertility and miscarriage prevention)
   a. Formulation 8 at least 50 mg bid
   b. Formulation 19 at least 50 mg QD
(2) a During second trimester
   a. Formulation 08 at least 100 mg bid
   b. Formulation 19 at least 100 mg QD
(3) During third trimester
   a. Formulation 09 at least 200 mg bid
   b. Formulation 19 at least 200 mg QD

The administration of the progesterone formulations can be used to treat and or prevent infertility, miscarriage, preterm labor and/or, preterm birth.

### Example 6

### Delay in Rise of Fetal Fibronectin Through Administration of Progesterone

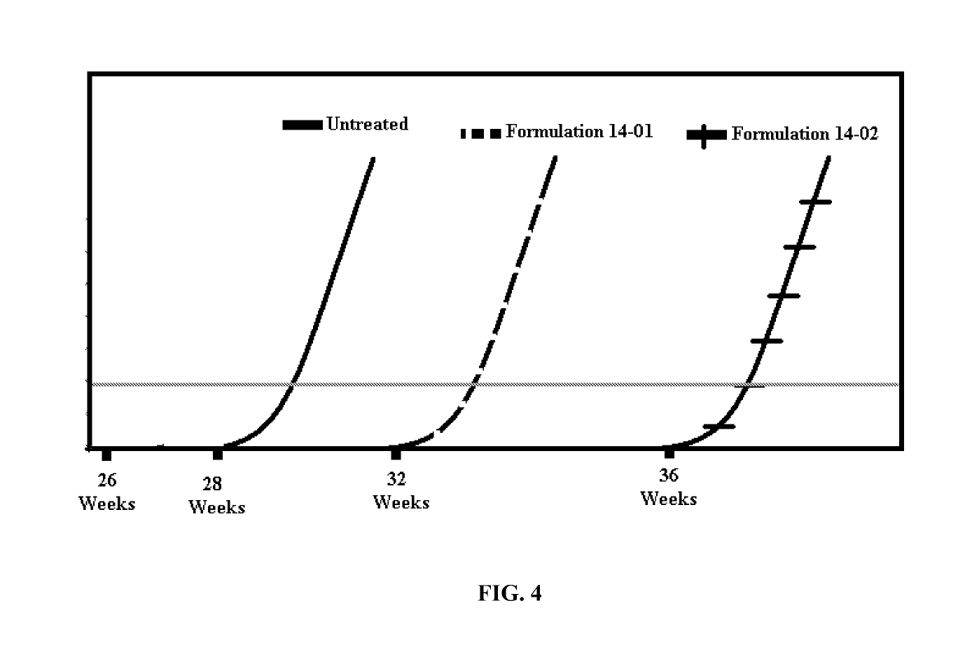

Three groups of women entering their 16 weeks of pregnancy are selected for the study. One group receives not medication (untreated), a second group receives 50 mg progesterone daily (1 capsule of Formulation 14), and a third group receives 100 mg progesterone daily (2 capsules of Example 14). The fetal fibronectin levels of each of the women participating in the study are measured daily. FIG. 4 shows a plot of projected fetal fibronectin levels of the pregnant women in the various groups. As can be seen in the figure, the rise of fetal fibronectin is delayed by the administration of the progesterone, with the larger amount of progesterone delaying the rise of fetal fibronectin for a longer period of time.

### Example 7

### Progesterone Dosing to Premenopausal Women

Two capsules of a commercial progesterone suspension product (Utrogestan) 100 mg/capsule and one dosage unit of Formulation 19 are administered premenopausal women with food, and pharmacokinetic testing carried out as described in Formulation 23. Additionally the metabolites were also monitored and pharmacokinetic parameters calculated. The results are shown in Table IX

TABLE IX

| | Cmax | |
|---|---|---|
| | 100 mg Utrogestan | Cmax* for |
| | (Besins) | Formulation 19 |
| Analyte | (nmol/L) | (nmol/L) |
| Progesterone (P) | 61 ± 37 | 357 |
| Pregnane Metabolite (PM) | 170 ± 44 | 228 |
| PM:P (ratio) | 2.79 | 0.64 |

*simulated.

For a therapeutic concentration of progesterone, PM: P Cₘₐₓ ratio with the formulation of the current invention is significantly lower compared to the commercial product. Therefore, the current inventive formulations should provide reduced adverse events such as sedation, dizziness and hypnosis. In the case of Formulation 19, it is postulated that 5α-reductase activity from the duodenum wall and 5β-reductase activity from the intestinal bacteria is blocked which may lead to lesser metabolism and higher progesterone value. In the case of controlled release dosage form minimal amount of progesterone is released at the upper GIT leading to lesser metabolism.

### Example 8

### Progesterone Dosing Regimen to Premenopausal Women

200 mg of progesterone are administered to premenopausal women using the formulation of Formulation 19 once-a-day administration. Similarly, 100 mg progesterone is administered using the Formulation of Example 17 administered twice-a-day. The Cₘₐₓ of the progesterone and the pregnane metabolites are measured and are shown in Table X

TABLE X

| | Formulation | Formulation |
|---|---|---|
| | 19 | 17 |
| | Cmax | Cmax |
| Analyte | (nmol/L) | (nmol/L) |
| Progesterone (P) | 565 | 400 |
| Pregnane Metabolite (PM) | 729 | 360 |
| PM:P (ratio) | 1.29 | 0.90 |

For a therapeutic concentration of progesterone, PM: P Cmax ratio with the formulation of the current invention is significantly lowered by adjusting the dosing regimen. Therefore, the inventive formulations should provide reduced adverse events such as sedation, dizziness and hypnosis.

It has to be understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

1. A pharmaceutical acceptable oral dosage form for pregnancy support, comprising:
a therapeutically effective amount of progesterone; and
a pharmaceutically acceptable carrier,
wherein after a single administration to a human subject, the oral dosage form produces pregnane metabolite mean Cₘₐₓ blood plasma level of less than about 1000 nmol/1.

2. The oral dosage form of claim 1, wherein the oral dosage form provides for progesterone Cₘₐₓ of about 175 ng/mL or less.

3. The oral dosage form of claim 1, wherein the oral dosage form is controlled release.

4. The oral dosage form of claim 1, wherein upon single dose administration to human subject produces a ratio of pregnane metabolite to progesterone mean Cₘₐₓ blood plasma level of less than 10.

5. The oral dosage form of claim 1, wherein pregnancy support is treatment for a condition selected from the group consisting of infertility, miscarriage, and pre term labor.

6. The oral dosage form of claim 1, wherein the oral dosage form includes about 25 mg to about 600 mg of progesterone.

7. The oral dosage form of claim 1, wherein the oral dosage form includes about 25 mg to about 95 mg of progesterone.

8. The oral dosage form of claim 1, wherein after a single administration to a human subject, the oral dosage form produces a ratio of mean plasma progesterone AUC to amount of progesterone administered of more than 1.5×10⁻⁶ hr/ml.

9. The oral dosage form of claim 1, wherein after single administration to a human subject, the oral dosage form produces a ratio of mean plasma progesterone AUC to amount of progesterone administered of more than 2.0×10⁻⁶ hr/ml.

10. The oral dosage form of claim 1, wherein the oral dosage form is substantially free of edible oils having a carbon chain length of 12 to 18 carbons.

11. The oral dosage form of claim 1, wherein the oral dosage form is substantially free of hydrophilic surfactants.

12. The oral dosage form of claim 1, wherein after a single administration to a female requiring pregnancy support during the first trimester of pregnancy, the oral dosage form provides an increase over endogenous baseline in the Cₘₐₓ of progesterone of at least 11 ng/ml.

13. The oral dosage form of claim 1, wherein after a single administration to a female requiring pregnancy support during the second trimester of pregnancy, the oral dosage form provides an increase over endogenous baseline in the Cₘₐₓ of progesterone of at least 25 ng/ml.

14. The oral dosage form of claim 1, wherein after a single administration to a female requiring pregnancy support during the third trimester of pregnancy, the oral dosage form provides an increase in the Cₘₐₓ of progesterone of at least 50 ng/ml.

15. The oral dosage form of claim 1, wherein after regular daily administration of the oral dosage form to a female requiring pregnancy support during the first trimester of pregnancy produces an increase in the steady state C_{avg} of progesterone of at least 11 ng/ml.

16. The oral dosage form of claim 1, wherein after regular daily administration of the oral dosage form to a female requiring pregnancy support during the first trimester the steady state C_{avg} of progesterone is less than 50 ng/mL.

17. The oral dosage form of claim 1, wherein after regular daily administration of the oral dosage form to a female requiring pregnancy support during the second trimester of pregnancy produces an increase in the steady state C_{avg} of progesterone of at least 25 ng/ml.

18. The oral dosage form of claim 1, wherein after regular daily administration of the oral dosage form to a female requiring pregnancy support during the third trimester of pregnancy produces an increase in the steady state C_{avg} of progesterone of at least 50 ng/ml.

19. The oral dosage form of claim 1, wherein the carrier is a release controlling agent.

20. The oral dosage form of claim 1, wherein the carrier includes at least one component selected from the group consisting of: celluloses; dextrins; gums; carbomers; methacrylates; sugars; lactoses; inorganic carbonates, oxides, chlorides sulphate and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

21. The oral dosage form of claim 1, wherein the carrier includes at least one component selected from the group consisting of celluloses; dextrins; gums; carbomers; methacrylates; inorganic carbonates; salts of calcium; salts of magnesium; fatty acids; fatty acid esters; gelatin; lactoses; polyethylene glycol; polyethylene oxide co-polymers; silicates; partially hydrogenated vegetable oils, fully hydrogenated vegetable oils, waxes, antioxidants, tocopherol, sugar stearates, starches, shellac, resins, proteins, and combinations thereof.

22. The oral dosage form of claim 1, wherein the carrier includes at least one component selected from the group consisting of: celluloses; dextrins; gums; carbomers; methacrylates; sugars; lactoses; inorganic carbonates; salts of calcium; salts of magnesium; Salts of fatty acids; inorganic and organic acids; bases and salts; propylene glycol; glycerols; fatty acids; fatty alcohols; fatty acid esters; glycerol esters; mono-, di-glycerol esters of fatty acids; omega oils; waxes; alcohols; gelatin; polyethylene glycol; polyethylene oxide co-polymers; silicates; antioxidants, tocopherol, sugar stearates, starches, shellac, resins, proteins, acrylates; methyl copolymers; polyvinyl alcohol; starch; phthalates; and combinations thereof.

23. A pharmaceutical oral dosage form for pregnancy support, comprising:
a therapeutically effective amount of progesterone, and
a pharmaceutically acceptable carrier,
wherein the oral dosage form has a dissolution rate in vitro, when measured using a USP Type-1 dissolution apparatus in 900 mL of deionized water with 2.0% (w/v) of sodium lauryl sulfate at 100 rpm, such that the oral dosage form releases at least 10 wt % of the progesterone in the first 30 minutes

24. The oral dosage form of claim 23, wherein the oral dosage form releases at least 10 wt % of the progesterone in the first 30 minutes.

25. The oral dosage form of claim 23, wherein the oral dosage form releases at least about 80 wt % of the progesterone after about 8 hours.

26. The oral dosage form of claim 23, wherein the oral dosage form includes about 25 mg to about 600 mg of progesterone.

27. The oral dosage form of claim 23, wherein the oral dosage form includes about 25 mg to about 95 mg of progesterone.

28. A pharmaceutical oral dosage form of claim 23 wherein after a single administration to a human subject, the oral dosage form produces a pregnane metabolite mean Cₘₐₓ blood plasma level of less than about 1000 nmol/1.

29. The oral dosage form of claim 23, wherein after a single administration to a human subject, the oral dosage form produces a ratio of mean plasma progesterone AUC to amount of progesterone administered of more than 1.5×10⁻⁶ hr/ml.

30. The oral dosage form of claim 23, wherein the oral dosage form is substantially free of edible oils having a carbon chain length of 12 to 18 carbons.

31. The oral dosage form of claim 23, wherein the oral dosage form is substantially free of hydrophilic surfactant.

32. The oral dosage form of claim 23, wherein after regular daily administration of the oral dosage form to a female during the first trimester of pregnancy produces an increase in the steady state C_{avg} of progesterone of at least 11 ng/ml.

33. The oral dosage form of claim 23, wherein after regular daily administration of the oral dosage form to a females during the second trimester of pregnancy produces an increase in the steady state C_{avg} of progesterone of at least 25 ng/ml.

34. The oral dosage form of claim 23 wherein after regular daily administration of the oral dosage form to a females during the third trimester of pregnancy produces an increase in the steady state C_{avg} of progesterone of at least 50 ng/ml.

35. The oral dosage form of claim 23, wherein the carrier includes at least one component selected from the group consisting of: celluloses; dextrins; gums; carbomers; methacrylates; sugars; lactoses; inorganic carbonates, oxides, chlorides sulphate and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

36. A pharmaceutical oral dosage form for pregnancy support, comprising:
a therapeutically effective amount of progesterone, and
a pharmaceutically acceptable carrier,
wherein the oral dosage form has a dissolution rate in vitro, when measured using a USP Type-1 dissolution apparatus in 900 mL of deionized water with 2.0% (w/v) of sodium lauryl sulfate at 100 rpm, such that the oral dosage form releases less than 45 wt % of the progesterone in the first 4 hours.

37. The oral dosage form of claim 36, wherein the oral dosage form releases at least 10 wt % of the progesterone in the first 30 minutes.

38. The oral dosage form of claim 36, wherein the oral dosage form releases at least about 80 wt % of the progesterone after about 8 hours.

39. The oral dosage form of claim 36, wherein the oral dosage form includes about 25 mg to about 600 mg of progesterone.

40. The oral dosage form of claim 36, wherein the oral dosage form includes about 25 mg to about 95 mg of progesterone.

41. A pharmaceutical oral dosage form of claim 36 wherein after a single administration to a human subject, the oral dosage form produces a pregnane metabolite mean Cₘₐₓ blood plasma level of less than about 1000 nmol/1.

42. The oral dosage form of claim 36, wherein after a single administration to a human subject, the oral dosage form produces a ratio of mean plasma progesterone AUC to amount of progesterone administered of more than 1.5×10⁻⁶ hr/ml.

43. The oral dosage form of claim 36, wherein the oral dosage form is substantially free of edible oils having a carbon chain length of 12 to 18 carbons.

44. The oral dosage form of claim 36, wherein the oral dosage form is substantially free of hydrophilic surfactant.

45. The oral dosage form of claim 36, wherein after regular daily administration of the oral dosage form to a female during the first trimester of pregnancy produces an increase in the steady state C_{avg} of progesterone of at least 11 ng/ml.

46. The oral dosage form of claim 36, wherein after regular daily administration of the oral dosage form to a females during the second trimester of pregnancy produces an increase in the steady state C_{avg} of progesterone of at least 25 ng/ml.

47. The oral dosage form of claim 36 wherein after regular daily administration of the oral dosage form to a females during the third trimester of pregnancy produces an increase in the steady state C_{avg} of progesterone of at least 50 ng/ml.

48. The oral dosage form of claim 36, wherein the carrier includes at least one component selected from the group consisting of: celluloses; dextrins; gums; carbomers; methacrylates; sugars; lactoses; inorganic carbonates, oxides, chlorides sulphate and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

49. A pharmaceutical oral dosage form, comprising:
a therapeutically effective amount of progesterone; and
a pharmaceutically acceptable carrier,
wherein after a single administration to a human subject, the oral dosage form produces a ratio of mean plasma progesterone AUC to the amount of progesterone administered of more than 1.5×10⁻⁶ hr/mL:1.

50. The oral dosage form of claim 49, wherein the oral dosage form provides a progesterone Cₘₐₓ of less than about 175 ng/ml.

51. The oral dosage form of claim 49, wherein the oral dosage form is a controlled release oral dosage form.

52. The oral dosage form of claim 49, wherein the oral dosage form includes about 10 mg to about 400 mg of progesterone.

53. The oral dosage form of claim 49, wherein the oral dosage form includes about 25 mg to 95 mg of progesterone.

54. The oral dosage form of claim 49, wherein after a single administration to a human subject, the oral dosage form produces a ratio of mean plasma progesterone AUC to the amount of progesterone administered of more than 1.5×10⁻⁶ hr/mL:1.

55. The oral dosage form of claim 49, wherein the oral dosage form is substantially free of edible oil having a carbon chain length of 12 to 18 carbons.

56. The oral dosage form of claim 49, wherein the oral dosage form is substantially free of hydrophilic surfactant.

57. The oral dosage form of claim 49, wherein the carrier includes at least one component selected from the group consisting of: celluloses; dextrins; gums; carbomers; methacrylates; sugars; lactoses; inorganic carbonates, oxides, chlorides sulphate and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

58. The oral dosage form of claim 49, wherein the carrier includes at least one component selected from the group consisting of celluloses; dextrins; gums; carbomers; methacrylates; inorganic carbonates; salts of calcium; salts of magnesium; fatty acids; fatty acid esters; gelatin; lactoses; polyethylene glycol; polyethylene oxide co-polymers; silicates; partially hydrogenated vegetable oils, fully hydrogenated vegetable oils, waxes, antioxidants, tocopherol, sugar stearates, starches, shellac, resins, proteins, and combinations thereof.

59. The oral dosage form of claim 49, wherein the carrier includes at least one component selected from the group consisting of: celluloses; dextrins; gums; carbomers; methacrylates; sugars; lactoses; inorganic carbonates; salts of calcium; salts of magnesium; salts of fatty acids; inorganic and organic acids; bases and salts; propylene glycol; glycerols; fatty acids; fatty alcohols; fatty acid esters; glycerol esters; mono-, di-glycerol esters of fatty acids; omega oils; waxes; alcohols; gelatin; polyethylene glycol; polyethylene oxide co-polymers; silicates; antioxidants, tocopherol, sugar stearates, starches, shellac, resins, proteins, acrylates; methyl copolymers; polyvinyl alcohol; starch; phthalates; and combinations thereof.

60. A method of delaying rise of at least 50 ng/ml of fetal fibronectin levels in a female requiring pregnancy support that is at least 16 weeks pregnant, comprising orally daily administering to the female at least 50 mg/day of progesterone.

61. The method of claim 60, wherein the rise in fetal fibronectin level is delayed for at least one week as compared to no progesterone treatment.

62. The method of claim 60, wherein the fetal fibronectin level is maintained below about 200 ng/ml.

63. The method of claim 60, wherein the fetal fibronectin level is maintained below about 50 ng/ml.

64. The method of claim 60, wherein the administration is done with an oral dosage form comprising, progesterone and a pharmaceutically acceptable carrier.

65. The method of claim 60, wherein the oral dosage form provides a progesterone Cₘₐₓ less than about 175 ng/ml.

66. The method of claim 60, wherein the oral dosage form includes about 25 mg to about 600 mg of progesterone.

67. The method of claim 60, wherein after a single administration to a human subject, the oral dosage form produces a ratio of mean plasma progesterone AUC to amount of progesterone administered of more than 1.5×10⁻⁶ hr/mL:1.

68. A method of reducing dizziness or sedation or both associated with the oral administration of progesterone comprising, administering an oral dosage form of claim 1, 23, 36 or 49, to a subject, wherein the administration reduces dizziness associated with the administration of the progesterone of claimed invention relative to a dosage form containing micronized progesterone suspended in peanut oil and which provides equivalent progesterone AUC values.

69. A method of treatment comprising:
administering an oral dosage form of any of claim 1, 23, 36, or 49, to a subject in need thereof, wherein the administration treats at least one condition selected from the group consisting of: preterm birth, preterm labor, infertility and miscarriage wherein the conditions based on their primary and secondary outcome measurements associated with the administration of the progesterone.
